(19) United States
(12) Patent Application Publication
(10) Pub. No.: US 2016/0324927 A1
(43) Pub. Date: Nov. 10, 2016

(54) COMPOSITIONS AND METHODS FOR IDENTIFYING, MODULATING AND MONITORING DRUG TARGETS IN MUSCULAR DISEASE

(71) Applicant: SomaLogic, Inc., Boulder, CO (US)

(72) Inventors: Larry Gold, Boulder, CO (US); Kirk DeLisle, Boulder, CO (US); David Sterling, Boulder, CO (US); Edward Brody, Boulder, CO (US); Britta Singer, Boulder, CO (US); Robert Mehler, Boulder, CO (US)

(21) Appl. No.: 15/144,593

(22) Filed: May 2, 2016

Related U.S. Application Data
(60) Provisional application No. 62/156,577, filed on May 4, 2015.

(51) Int. Cl. A61K 38/18; G01N 33/68; A61K 45/06

(52) U.S. Cl. CPC A61K 38/1875; A61K 45/06; G01N 33/6893; G01N 2800/2885; G01N 2800/52; G01N 2800/56

(57) ABSTRACT

The present disclosure relates to customized therapy for disease. The present disclosure also relates to aptamer-based compositions and methods for identifying, modulating and monitoring drug targets in muscular disease (e.g., Duchenne muscular dystrophy).

### CROSS REFERENCE TO RELATED APPLICATION

The present application claims priority to U.S. Provisional Application No. 62/156,577, filed May 4, 2015, which is incorporated by reference herein in its entirety for any purpose.

### FIELD OF THE INVENTION

The present invention relates to customized therapy for disease. In particular, the present invention relates to aptamer-based compositions and methods for identifying, modulating and monitoring drug targets in muscular disease (e.g., Duchenne muscular dystrophy).

### BACKGROUND OF THE INVENTION

Muscle diseases include many diseases and ailments that either directly, via intrinsic muscle pathology, or indirectly, via nerve or neuromuscular junction pathology, impair the functioning of the muscles. Muscular dystrophy (MD) is a group of muscle diseases that weaken the musculoskeletal system and hamper locomotion. Muscular dystrophies are characterized by progressive skeletal muscle weakness, defects in muscle proteins, and the death of muscle cells and tissue.

Duchenne muscular dystrophy (DMD) is a severe form of myopathy with an incidence of about 1 in 3,600 to 9,337 boys worldwide. The disease is a result of different types of mutations in the X-linked DMD gene that abolish the expression and/or biological activity of dystrophin, an essential protein for muscle fiber plasma membrane integrity and myofiber function. Clinically, the disease is characterized by progressive muscle wasting leading to loss of ambulation by 8-15 years of age and early death from complications from respiratory, orthopedic, and cardiac problems.

Several current drug development programs are focused on slowing or preventing the progressive muscle loss in DMD either in conjunction with the standard of care or as a stand-alone therapy. Standard of care is currently chronic high-dose glucocorticoids, which are able to slow disease progression by only a few years, but are associated with a significant array of side effects. Promising therapeutic approaches for DMD include restoring expression of the dystrophin via exon skipping strategies, viral-based gene therapies, and nonsense suppression/read-through strategies. Other genetic approaches include delivering mini-dystrophins, upregulation of utrophin to compensate for the missing dystrophin, and many others. Pharmacological strategies include corticosteroid dissociative drugs with greater efficacy and with few or no side effects, other anti-inflammatory therapies, and effectors of signaling pathways. The accepted primary clinical endpoint used for determining efficacy in the majority of these therapeutic approaches for ambulatory boys with DMD is the “six minute walk test”. What all of these drug development programs lack is a reliable surrogate biomarker or set of biomarkers, ideally based on measurable molecules, to accurately gauge progression of the disease, to determine efficacy of treatment, and to determine when a therapy will be most effective in both ambulatory and non-ambulatory boys with DMD. Moreover, there exists a great need for further therapeutic options for DMD patients.

### SUMMARY OF THE INVENTION

The present invention relates to customized therapy for disease. In particular, the present invention relates to aptamer-based compositions and methods for identifying, modulating and monitoring drug targets in muscular disease (e.g., Duchenne muscular dystrophy).

For example, in some embodiments, the present invention provides a method for identifying protein targets, comprising: a) assaying a biological sample from a subject diagnosed with a disease to identify altered levels of one or more proteins relative to the level of the protein in a reference sample; and b) identifying one or more treatments that targets one or more of the proteins with altered expression. The present invention is not limited to particular protein targets. In some embodiments, targets are identified by screening samples for levels of protein expression and comparing the levels to normal (e.g., disease-free) tissue (e.g., using technologies described herein (e.g., aptamer technology described herein)). The invention is not limited by the target identified (e.g., using aptamer technology described herein). In some embodiments, the reference sample is a sample of normal tissue from the subject, or a population average of normal tissue. In some embodiments, the level of the proteins are altered at least 2-fold (e.g., at least 4-fold, at least 5-fold, at least 10-fold, at least 20-fold, at least 50-fold, at least 100-fold, or more). In some embodiments, the method further comprises the step of administering the one or more treatments to the subject. In some embodiments, the method further comprises the step of determining the presence of mutations in the proteins. In some embodiments, the disease is, for example, a muscular disease (e.g., DMD or other muscular disease described herein.), a genetic disease, a metabolic disorder, an inflammatory disease, or an infectious disease. In some embodiments, the biological sample is selected from, for example, tissue, whole blood, leukocytes, peripheral blood mononuclear cells, buffy coat, plasma, serum, sputum, tears, mucus, nasal washes, nasal aspirate, breath, urine, semen, saliva, peritoneal washings, ascites, cystic fluid, meningeal fluid, amniotic fluid, glandular fluid, pancreatic fluid, lymph fluid, pleural fluid, cytologic fluid, nipple aspirate, bronchial aspirate, bronchial brushing, synovial fluid, joint aspirate, organ secretions, cells, a cellular extract, or cerebrospinal fluid. In some embodiments, the drug is, for example, those described herein. In some embodiments, the assaying comprises contacting the sample with a plurality of aptamers specific for the proteins.

Further embodiments provide a method for determining a treatment course of action, comprising: a) assaying a tissue sample from a subject diagnosed with a muscular disease (e.g., DMD) to identify altered levels of one or more proteins (e.g., Growth differentiation factor 11 (GDF-11), Receptor Expressed in Lymphoid Tissues (RELT), Complement decay-accelerating factor (CD55), WAP, kazal, immunoglobulin, kunitz and NTR domain-containing protein 1 (WFIKKN1), gelsolin, and/or other protein identified utilizing the compositions and methods of the invention (e.g., described in Example 1)), relative to the level of the proteins in normal tissue (e.g., normal subject without muscular disease); and b) administering one or more treatments that targets one or more of the proteins with altered expression.

Additional embodiment provide a method for treating a disease, comprising: a) assaying a biological sample from a subject diagnosed with a disease to identify altered levels of one or more proteins relative to the level of the protein in a reference sample; and b) administering one or more treatments that target one or more of the proteins with altered expression to the subject.

Further embodiment provide a method for treating a disease, comprising: a) assaying a biological sample from a subject diagnosed with a disease to identify altered levels of one or more proteins relative to the level of the protein in a reference sample; and b) administering one or more treatments that target one or more of the proteins with altered expression to the subject; and c) repeating the step of assaying the biological sample from a subject diagnosed with a disease to identify altered levels of one or more proteins relative to the level of the protein in a reference sample.

Yet other embodiments provide a method for monitoring treating of a disease, comprising: a) assaying a biological sample from a subject diagnosed with a disease to identify altered levels of one or more proteins relative to the level of the protein in a reference sample; b) administering one or more treatments that target one or more of the proteins with altered expression to the subject; and c) repeating step a) one or more times.

Still further embodiments provide a method for screening test compounds, comprising: a) assaying a biological sample from a subject diagnosed with a disease to identify altered levels of one or more proteins relative to the level of the protein in a reference sample; b) administering one or more test compounds that target or are suspected of targeting one or more of the proteins with altered expression to the subject; and c) repeating step a) one or more times.

In another aspect, the invention relates to a method of treating a muscle disease, comprising the step of administering to a mammal in need thereof a therapeutically effective amount of Growth differentiation factor 11 (GDF-11), Receptor Expressed in Lymphoid Tissues (RELT), Complement decay-accelerating factor (CD55), WAP, kazal, immunoglobulin, kunitz and NTR domain-containing protein 1 (WFIKKN1), gelsolin, fibroblast activation protein alpha (FAP), protein jagged-1 (JAG1), bone sialoprotein 2 (IBSP), ADAM metallopeptidase domain 9 (ADAM9), cadherin-5 (CDH5), neural cell adhesion molecule L1-like protein (CHL1), osteomodulin (OMD), contactin-5 (CNTN5), and/or other protein identified utilizing the compositions and methods of the invention (e.g., described in Example 1). In some embodiments, a method comprises treating a muscle disease comprising administering GDF-11. In some embodiments the method further comprises administering an antagonist of GDF-8. In some embodiments, the disease is Duchenne muscular dystrophy.

Another aspect of the invention relates to a method of improving normal muscle function, comprising the step of administering to a mammal in need thereof a therapeutically effective amount of GDF-11, RELT, CD55, WFIKKN1, gelsolin, fibroblast activation protein alpha (FAP), protein jagged-1 (JAG1), bone sialoprotein 2 (IBSP), ADAM metallopeptidase domain 9 (ADAM9), cadherin-5 (CDH5), neural cell adhesion molecule L1-like protein (CHL1), osteomodulin (OMD), contactin-5 (CNTN5), and/or other protein identified utilizing the compositions and methods of the invention (e.g., described in Example 1).

Yet another aspect of the invention relates to a method of upregulating GDF-11, RELT, CD55, WFIKKN1, gelsolin, fibroblast activation protein alpha (FAP), protein jagged-1 (JAG1), bone sialoprotein 2 (IBSP), ADAM metallopeptidase domain 9 (ADAM9), cadherin-5 (CDH5), neural cell adhesion molecule L1-like protein (CHL1), osteomodulin (OMD), contactin-5 (CNTN5), and/or other protein identified utilizing the compositions and methods of the invention (e.g., described in Example 1). Another aspect of the invention relates to a method of upregulating a target of GDF-11, RELT, CD55, WFIKKN1, gelsolin, fibroblast activation protein alpha (FAP), protein jagged-1 (JAG1), bone sialoprotein 2 (IBSP), ADAM metallopeptidase domain 9 (ADAM9), cadherin-5 (CDH5), neural cell adhesion molecule L1-like protein (CHL1), osteomodulin (OMD), contactin-5 (CNTN5), and/or other protein identified utilizing the compositions and methods of the invention (e.g., described in Example 1), comprising the step of administering to a mammal in need thereof a therapeutically effective amount of GDF-11, RELT, CD55, WFIKKN1, gelsolin, fibroblast activation protein alpha (FAP), protein jagged-1 (JAG1), bone sialoprotein 2 (IBSP), ADAM metallopeptidase domain 9 (ADAM9), cadherin-5 (CDH5), neural cell adhesion molecule L1-like protein (CHL1), osteomodulin (OMD), contactin-5 (CNTN5) and/or other protein identified utilizing the compositions and methods of the invention.

In one embodiment, the present invention provides a method of treating Duchenne Muscular Dystrophy (DMD) including administering to an individual who is suffering from or susceptible to DMD an effective amount of a recombinant GDF-11, RELT, CD55, WFIKKN1, gelsolin, fibroblast activation protein alpha (FAP), protein jagged-1 (JAG1), bone sialoprotein 2 (IBSP), ADAM metallopeptidase domain 9 (ADAM9), cadherin-5 (CDH5), neural cell adhesion molecule L1-like protein (CHL1), osteomodulin (OMD), contactin-5 (CNTN5) and/or other protein identified utilizing the compositions and methods of the invention (e.g., described in Example 1) such that at least one symptom or feature of DMD is reduced in intensity, severity, or frequency, or has delayed onset. In some embodiments, at least one symptom or feature of DMD is selected from muscle wasting, muscle weakness, muscle fragility, joint contracture, skeletal deformation, fatty infiltration of muscle, replacement of muscle with non-contractile tissue (e.g., muscle fibrosis), muscle necrosis, cardiomyopathy, impaired swallowing, impaired bowel and bladder function, muscle ischemia, cognitive impairment function (e.g., learning difficulties, higher risk of neurobehavioral disorders, cognitive defects), behavioral dysfunction, socialization impairment, scoliosis, and/or impaired respiratory function.

In some embodiments, the present invention provides methods of identifying an efficacious muscular dystrophy therapy (e.g., Duchenne muscular dystrophy therapy) for a subject comprising: a) testing a sample from a subject to determine the level of at least one biomarker selected from GDF-11, RELT, CD55, gelsolin, WFIKKN1, fibroblast activation protein alpha (FAP), protein jagged-1 (JAG1), bone sialoprotein 2 (IBSP), ADAM metallopeptidase domain 9 (ADAM9), cadherin-5 (CDH5), neural cell adhesion molecule L1-like protein (CHL1), osteomodulin (OMD), contactin-5 (CNTN5) and/or other protein identified utilizing the compositions and methods of the invention (e.g., described in Example 1); and b) identifying a muscular dystrophy therapy that is effective for treating muscular dystrophy (e.g., Duchenne muscular dystrophy) in the subject based on the level of the at least one biomarker that is determined.

In certain embodiments, the at least one biomarker comprises GDF-11, RELT, CD55, gelsolin, WFIKKN1, fibroblast activation protein alpha (FAP), protein jagged-1 (JAG1), bone sialoprotein 2 (IBSP), ADAM metallopeptidase domain 9 (ADAM9), cadherin-5 (CDH5), neural cell adhesion molecule L1-like protein (CHL1), osteomodulin (OMD), contactin-5 (CNTN5) and/or other protein identified utilizing the compositions and methods of the invention (e.g., described in Example 1) and the therapy comprises a therapeutic that targets the one or more biomarkers. In further embodiments, the therapy is administered to the subject. In certain embodiments, the sample comprises a blood sample, plasma sample, or urine sample (or any other biological sample) from the subject.

In particular embodiments, the present invention provides methods of identifying the presence, severity, or risk of exacerbation of muscular dystrophy (e.g., Duchenne muscular dystrophy) in a subject comprising: a) testing a sample from a subject to determine the level of at least one biomarker selected from GDF-11, RELT, CD55, gelsolin, WFIKKN1, fibroblast activation protein alpha (FAP), protein jagged-1 (JAG1), bone sialoprotein 2 (IBSP), ADAM metallopeptidase domain 9 (ADAM9), cadherin-5 (CDH5), neural cell adhesion molecule L1-like protein (CHL1), osteomodulin (OMD), contactin-5 (CNTN5) and/or other protein identified utilizing the compositions and methods of the invention (e.g., described in Example 1); and b) identifying the presence, severity, or risk of exacerbation of muscular dystrophy in the subject based on an elevated or decreased level of the at least one biomarker. In some embodiments, the method comprises assaying a first biological sample from a subject with a muscular disease and assaying a second biological sample from the subject, wherein the first and second biological samples were taken at a first time point and a second time point, to identify altered levels of one or more proteins listed in Table 2 at the second time point relative to the first time point. In some embodiments, a decrease in the level of at least one protein selected from GDF-11, RELT, CD55, WFIKKN1, gelsolin, fibroblast activation protein alpha (FAP), protein jagged-1 (JAG1), bone sialoprotein 2 (IBSP), ADAM metallopeptidase domain 9 (ADAM9), and cadherin-5 (CDH5), neural cell adhesion molecule L1-like protein (CHL1), osteomodulin (OMD), and contactin-5 (CNTN5) at the second time point relative to the first time point, or an increase in the levels of at least one protein selected from HSPA1A, MAPK12, CAMK2A, CXCL10, RET, and persephin at the second time point relative to the first time point, indicates that the muscular disease has progressed between the first time point and the second time point. In some embodiments, an increase in the level of at least one protein selected from GDF-11, RELT, CD55, WFIKKN1, gelsolin, fibroblast activation protein alpha (FAP), protein jagged-1 (JAG1), bone sialoprotein 2 (IBSP), ADAM metallopeptidase domain 9 (ADAM9), and cadherin-5 (CDH5) at the second time point relative to the first time point, or a decrease in the levels of at least one protein selected from contactin-5 (CNTN5), CXCL10, RET, and persephin at the second time point relative to the first time point, indicates that the muscular disease has improved between the first time point and the second time point.

In certain embodiments, the methods further comprise administering a therapy to the subject. In other embodiments, the methods further comprise informing the subject that they have muscular dystrophy, the severity of the muscular dystrophy, and/or the risk of exacerbating the muscular dystrophy.

In some embodiments, the present invention provides methods of monitoring response to muscular dystrophy therapy comprising: a) testing a sample from a subject receiving muscular dystrophy therapy to determine the level of at least one biomarker selected from GDF-11, RELT, CD55, gelsolin, WFIKKN1, fibroblast activation protein alpha (FAP), protein jagged-1 (JAG1), bone sialoprotein 2 (IBSP), ADAM metallopeptidase domain 9 (ADAM9), cadherin-5 (CDH5), neural cell adhesion molecule L1-like protein (CHL1), osteomodulin (OMD), contactin-5 (CNTN5), HSPA1A, MAPK12, CAMK2A, CXCL10, RET, and persephin and/or other protein identified utilizing the compositions and methods of the invention (e.g., described in Example 1); and b) adjusting, or continuing un-adjusted, the therapy based on the level of the at least one biomarker that is determined.

In certain embodiments, the present invention provides methods of treating muscular dystrophy comprising: administering to a subject with muscular dystrophy an agent that inhibits (e.g., inhibits nucleic acid or protein expression or activity) at least one biomarker selected from GDF-11, RELT, CD55, gelsolin, WFIKKN1, fibroblast activation protein alpha (FAP), protein jagged-1 (JAG1), bone sialoprotein 2 (IBSP), ADAM metallopeptidase domain 9 (ADAM9), cadherin-5 (CDH5), neural cell adhesion molecule L1-like protein (CHL1), osteomodulin (OMD), and contactin-5 (CNTN5) and/or other protein identified utilizing the compositions and methods of the invention (e.g., described in Example 1). The invention is not limited to any particular agent inhibitor. Indeed, any agent known in the art that inhibits nucleic acid or protein expression or activity can be used including, but not limited to, an antibody or fragment thereof, small molecule, antisense, siRNA, or micro-RNA. In other embodiments, the present invention provides methods of treating muscular dystrophy comprising: administering to a subject with muscular dystrophy an agent that activates at least one biomarker selected from GDF-11, RELT, CD55, gelsolin, WFIKKN1, fibroblast activation protein alpha (FAP), protein jagged-1 (JAG1), bone sialoprotein 2 (IBSP), ADAM metallopeptidase domain 9 (ADAM9), cadherin-5 (CDH5), neural cell adhesion molecule L1-like protein (CHL1), osteomodulin (OMD), and contactin-5 (CNTN5) and/or other protein identified utilizing the compositions and methods of the invention (e.g., described in Example 1). The invention is not limited to any particular agent activator. Indeed, any agent known in the art that activates at least one biomarker described herein may be used.

### BRIEF DESCRIPTION OF THE DRAWINGS

FIGS. 1A-F depicts that Cumulative Distribution Function (CDF) plots for six exemplary proteins from the combined cohort analysis identified in Example 1, including proteins that are increased (A, Troponin I, fast skeletal muscle; B, myoglobin; C, heat shock protein 70) and decreased (D, RET; E, gelsolin; F, bone sialoprotein 2) in DMD patients vs. controls.

FIG. 2A-D shows exemplary proteins from four “types” of age-related changes in protein signal levels seen in DMD patients vs. controls in this study. 3A, creatine kinase; 3B, RET; 3C, Phospholipase A2, Group IIA; 3D, growth-differentiation factor detailed description.

The present invention relates to customized therapy for disease. In particular, the present invention relates to aptamer-based compositions and methods for identifying, modulating and monitoring drug targets in muscular disease (e.g., Duchenne muscular dystrophy).

### I. Definitions

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

In order to facilitate review of the various embodiments of the disclosure, the following explanations of specific terms are provided:

Aptamer: The term aptamer, as used herein, refers to a non-naturally occurring nucleic acid that has a desirable action on a target molecule. A desirable action includes, but is not limited to, binding of the target, catalytically changing the target, reacting with the target in a way that modifies or alters the target or the functional activity of the target, covalently attaching to the target (as in a suicide inhibitor), and facilitating the reaction between the target and another molecule.

Analog: The term analog, as used herein, refers to a structural chemical analog as well as a functional chemical analog. A structural chemical analog is a compound having a similar structure to another chemical compound but differing by one or more atoms or functional groups. This difference may be a result of the addition of atoms or functional groups, absence of atoms or functional groups, the replacement of atoms or functional groups or a combination thereof. A functional chemical analog is a compound that has similar chemical, biochemical and/or pharmacological properties. The term analog may also encompass S and R steroisomers of a compound.

Bioactivity: The term bioactivity, as used herein, refers to one or more intercellular, intracellular or extracellular process (e.g., cell-cell binding, ligand-receptor binding, cell signaling, etc.) which can impact physiological or pathophysiological processes.

C-5 Modified Pyrimidine: C-5 modified pyrimidine, as used herein, refers to a pyrimidine with a modification at the C-5 position. Examples of a C-5 modified pyrimidine include those described in U.S. Pat. Nos. 5,719,273 and 5,945,527. Additional examples are provided herein.

Consensus Sequence: Consensus sequence, as used herein, refers to a nucleotide sequence that represents the most frequently observed nucleotide found at each position of a series of nucleic acid sequences subject to a sequence alignment.

Covalent Bond: Covalent bond or interaction refers to a chemical bond that involves the sharing of at least a pair of electrons between atoms.

Modified: The term modified (or modify or modification) and any variations thereof, when used in reference to an oligonucleotide, means that at least one of the four constituent nucleotide bases (i.e., A, G, T/U, and C) of the oligonucleotide is an analog or ester of a naturally occurring nucleotide.

Modulate: The term modulate, as used herein, means to alter the expression level of a peptide, protein or polypeptide by increasing or decreasing its expression level relative to a reference expression level, and/or alter the stability and/or activity of a peptide, protein or polypeptide by increasing or decreasing its stability and/or activity level relative to a reference stability and/or activity level.

Non-covalent Bond: Non-covalent bond or non-covalent interaction refers to a chemical bond or interaction that does not involve the sharing of pairs of electrons between atoms. Examples of non-covalent bonds or interactions includes hydrogen bonds, ionic bonds (electrostatic bonds), van der Waals forces and hydrophobic interactions.

Nucleic Acid: Nucleic acid, as used herein, refers to any nucleic acid sequence containing DNA, RNA and/or analogs thereof and may include single, double and multi-stranded forms. The terms “nucleic acid”, “oligo”, “oligonucleotide” and “polynucleotide” may be used interchangeably.

Pharmaceutically Acceptable: Pharmaceutically acceptable, as used herein, means approved by a regulatory agency of a federal or a state government or listed in the U.S. Pharmacopoeia or other generally recognized pharmacopoeia for use in animals and, more particularly, in humans.

Pharmaceutically Acceptable Salt: Pharmaceutically acceptable salt or salt of a compound (e.g., aptamer), as used herein, refers to a product that contains an ionic bond and is typically produced by reacting the compound with either an acid or a base, suitable for administering to an individual. A pharmaceutically acceptable salt can include, but is not limited to, acid addition salts including hydrochlorides, hydrobromides, phosphates, sulphates, hydrogen sulphates, alkylsulphonates, arylsulphonates, arylalkylsulfonates, acetates, benzoates, citrates, maleates, fumarates, succinates, lactates, and tartrates; alkali metal cations such as Li, Na, K, alkali earth metal salts such as Mg or Ca, or organic amine salts.

Pharmaceutical Composition: Pharmaceutical composition, as used herein, refers to formulation comprising a pharmaceutical agent (e.g., drug) in a form suitable for administration to an individual. A pharmaceutical composition is typically formulated to be compatible with its intended route of administration. Examples of routes of administration include, but are not limited to, oral and parenteral, e.g., intravenous, intradermal, subcutaneous, inhalation, topical, transdermal, transmucosal, and rectal administration.

SELEX: The term SELEX, as used herein, refers to generally to the selection for nucleic acids that interact with a target molecule in a desirable manner, for example binding with high affinity to a protein; and the amplification of those selected nucleic acids. SELEX may be used to identify aptamers with high affinity to a specific target molecule. The term SELEX and “SELEX process” may be used interchangeably.

Sequence Identity: Sequence identity, as used herein, in the context of two or more nucleic acid sequences is a function of the number of identical nucleotide positions shared by the sequences (i.e., % identity=number of identical positions/total number of positions×100), taking into account the number of gaps, and the length of each gap that needs to be introduced to optimize alignment of two or more sequences. The comparison of sequences and determination of percent identity between two or more sequences can be accomplished using a mathematical algorithm, such as BLAST and Gapped BLAST programs at their default parameters (e.g., Altschul et al., J. Mol. Biol. 215:403, 1990; see also BLASTN at www.ncbi.nlm.nih.gov/BLAST). For sequence comparisons, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters. Optimal alignment of sequences for comparison can be conducted, e.g., by the local homology algorithm of Smith and Waterman, Adv. Appl. Math., 2:482, 1981, by the homology alignment algorithm of Needleman and Wunsch, J. Mol. Biol., 48:443, 1970, by the search for similarity method of Pearson and Lipman, Proc. Nat'l. Acad. Sci. USA 85:2444, 1988, by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally, Ausubel, F. M. et al., Current Protocols in Molecular Biology, pub. by Greene Publishing Assoc. and Wiley—Interscience (1987)). As used herein, when describing the percent identity of a nucleic acid, such as an aptamer, the sequence of which is at least, for example, about 95% identical to a reference nucleotide sequence, it is intended that the nucleic acid sequence is identical to the reference sequence except that the nucleic acid sequence may include up to five point mutations per each 100 nucleotides of the reference nucleic acid sequence. In other words, to obtain a desired nucleic acid sequence, the sequence of which is at least about 95% identical to a reference nucleic acid sequence, up to 5% of the nucleotides in the reference sequence may be deleted or substituted with another nucleotide, or some number of nucleotides up to 5% of the total number of nucleotides in the reference sequence may be inserted into the reference sequence (referred to herein as an insertion). These mutations of the reference sequence to generate the desired sequence may occur at the 5′ or 3′ terminal positions of the reference nucleotide sequence or anywhere between those terminal positions, interspersed either individually among nucleotides in the reference sequence or in one or more contiguous groups within the reference sequence.

SOMAmer: The term SOMAmer, as used herein, refers to an aptamer having improved off-rate characteristics. SOMAmers are alternatively referred to as Slow Off-Rate Modified Aptamers, and may be selected via the improved SELEX methods described in U.S. Publication No. 20090004667, entitled “Method for Generating Aptamers with Improved Off-Rates”, which is incorporated by reference in its entirety.

Spacer Sequence: Spacer sequence, as used herein, refers to any sequence comprised of small molecule(s) covalently bound to the 5′-end, 3′-end or both 5′ and 3′ ends of the nucleic acid sequence of an aptamer. Exemplary spacer sequences include, but are not limited to, polyethylene glycols, hydrocarbon chains, and other polymers or copolymers that provide a molecular covalent scaffold connecting the consensus regions while preserving aptamer binding activity. In certain aspects, the spacer sequence may be covalently attached to the aptamer through standard linkages such as the terminal 3′ or 5′ hydroxyl, 2′ carbon, or base modification such as the C5-position of pyrimidines, or C8 position of purines.

Target Molecule: Target molecule (or target), as used herein, refers to any compound or molecule upon which a nucleic acid can act in a desirable manner (e.g., binding of the target, catalytically changing the target, reacting with the target in a way that modifies or alters the target or the functional activity of the target, covalently attaching to the target (as in a suicide inhibitor), and facilitating the reaction between the target and another molecule). Non-limiting examples of a target molecule include a protein, peptide, nucleic acid, carbohydrate, lipid, polysaccharide, glycoprotein, hormone, receptor, antigen, antibody, virus, pathogen, toxic substance, substrate, metabolite, transition state analog, cofactor, inhibitor, drug, dye, nutrient, growth factor, cell, tissue, any portion or fragment of any of the foregoing, etc. Virtually any chemical or biological effector may be a suitable target. Molecules of any size can serve as targets. A target can also be modified in certain ways to enhance the likelihood or strength of an interaction between the target and the nucleic acid. A target may also include any minor variation of a particular compound or molecule, such as, in the case of a protein, for example, variations in its amino acid sequence, disulfide bond formation, glycosylation, lipidation, acetylation, phosphorylation, or any other manipulation or modification, such as conjugation with a labeling component, which does not substantially alter the identity of the molecule. A “target molecule” or “target” is a set of copies of one type or species of molecule or multimolecular structure that is capable of binding to an aptamer. “Target molecules” or “targets” refer to more than one such set of molecules.

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. “Comprising A or B” means including A, or B, or A and B. It is further to be understood that all base sizes or amino acid sizes, and all molecular weight or molecular mass values, given for nucleic acids or polypeptides are approximate, and are provided for description.

Further, ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (as well as fractions thereof unless the context clearly dictates otherwise). Any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, are to be understood to include any integer within the recited range, unless otherwise indicated. As used herein, “about” or “consisting essentially of mean±20% of the indicated range, value, or structure, unless otherwise indicated. As used herein, the terms “include” and “comprise” are open ended and are used synonymously. It should be understood that the terms “a” and “an” as used herein refer to “one or more” of the enumerated components. The use of the alternative (e.g., “or”) should be understood to mean either one, both, or any combination thereof of the alternatives

Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including explanations of terms, will control. In addition, the materials, methods, and examples are illustrative only and not intended to be limiting.

### II. Methods for Identifying, Modulating and Monitoring Drug Targets in Muscular Disease

Growth differentiation factor 11 (GDF11) also known as bone morphogenetic protein 11 (BMP-11) is a protein that in humans is encoded by the GDF11 gene. This BMP group of proteins is characterized by a polybasic proteolytic processing site, which is cleaved to produce a protein containing seven conserved cysteine residues. GDF11 is a myostatin-homologous protein that acts as an inhibitor of nerve tissue growth. GDF11 has been shown to suppress neurogenesis through a pathway similar to that of myostatin, including stopping the progenitor cell-cycle during G-phase. The similarities between GDF11 and myostatin imply a likelihood that the same regulatory mechanisms are used to control tissue size during both muscular and neural development.

GDF11 belongs to the transforming growth factor beta superfamily that controls anterior-posterior patterning by regulating the expression of Hox genes. It determines Hox gene expression domains and rostrocaudal identity in the caudal spinal cord.

During mouse development, GDF11 expression begins in the tail bud and caudal neural plate region. GDF knock-out mice display skeletal defects as a result of patterning problems with anterior-posterior positioning. Peripheral supplementation of GDF11 protein (in mice) ameliorates the age-related dysfunction of skeletal muscle by rescuing the function of aged muscle stem cells.

This cytokine also inhibits the proliferation of olfactory receptor neuron progenitors to regulate the number of olfactory receptor neurons occurring in the olfactory epithelium, and controls the competence of progenitor cells to regulate numbers of retinal ganglionic cells developing in the retina. Other studies in mice suggest that GDF11 is involved in mesodermal formation and neurogenesis during embryonic development. The members of this TGF-β superfamily are involved in the regulation of cell growth and differentiation not only in embryonic tissues, but adult tissues as well.

GDF11 can bind type I TGF-beta superfamily receptors ACVR1B (ALK4), TGFBR1 (ALK5) and ACVR1C (ALK7), but predominantly uses ALK4 and ALK5 for signal transduction.

GDF11 is closely related to myostatin, a negative regulator of muscle growth. Both myostatin and GDF11 are involved in the regulation of cardiomyocyte proliferation. GDF11 is also a negative regulator of neurogenesis, the production of islet progenitor cells, the regulation of kidney organogenesis, pancreatic development, the rostro-caudal patterning in the development of spinal cords, and is a negative regulator of chondrogenesis. Due to the similarities between myostatin and GDF11, the actions of GDF11 are likely regulated by WFIKKN2, a large extracellular multidomain protein consisting of follistatin, immunoglobulin, protease inhibitor, and NTR domains. WFIKKN2 has a high affinity for GDF11, and previously has been found to inhibit the biological activities of myostatin.

Duchenne Muscular Dystrophy is the result of a deficient dystrophin gene. The dystrophin gene is encoded on the X chromosome, and thus women are effectively diploid for dystrophin (even though the condensed X chromosome—Barr bodies—are randomly and stochastically distributed in the cells of a woman and thus individual cells have either the normal level of dystrophin or none at all. On average, a woman carrier with a single dystrophin null mutation has roughly half the level of the dystrophin protein as a non-carrier woman. The fact that carrier women are largely (but not always) without the symptoms of DMD suggests that the dystrophin protein will suffice for health at half the normal concentration.

Most therapeutic attempts at treatments for DMD are dystrophin-centric; that is, the pharmaceutical industry and academic clinical researchers have focused on methods to provide some non-mutant dystrophin to boys with DMD. Many attempts have been made to introduce DNA encoding dystrophin and smaller (but functional) variants of dystrophin into muscle cells lacking a functional copy of dystrophin. These experiments have largely failed. Other attempts have been made to cause the splicing of the dystrophin mRNA to “skip” a deleterious dystrophin mutation, thus allowing a functional (but truncated) dystrophin to be expressed. These experiments are also difficult and have not yet led to amelioration of the DMD phenotype. Perhaps the most successful clinical trials for DMD have been carried out by the biotech company PTC Therapeutics, using their drug ATALUREN, a drug that causes ribosomes to “read through” nonsense codons in the dystrophin mRNA and thus provides some fully functional dystrophin to DMD patients. ATALUREN will only be useful for the ˜10% of the DMD patients that have a nonsense codon in dystrophin as the inactivating mutation.

Gene therapy, exon skipping, and nonsense suppression are three different examples of dystrophin-centric therapies; each is aimed at restoring some quantity of normal (or adequately functional) dystrophin protein to DMD patients who have no functional protein whatsoever. Each approach presents unique difficulties.

A complementary approach to treatment of DMD patients is to attempt to stimulate the muscle cells of a DMD patient to become more functional in the absence of dystrophin. There have been attempts in pre-clinical studies to all DMD patient to become more functional in the absence of dystrophin via causing an embryonic version of dystrophin to continue to be expressed in older patients. These approaches have yet to show efficacy. Such an approach may be referred to as a near-dystrophin-centric or dystrophin-homologue-centric approach.

In a different approach, therapy for DMD patients can focus on stimulating muscle cell function in the absence of dystrophin through injections of a drug that stimulates muscle cell function. In one aspect, the drug would function on the outside of muscle cells, rather than as an obligate intracellular compound (like dystrophin).

Thus, experiments were conducted during development of embodiments of the invention in order to identify protein targets for disease (e.g., muscular disease (e.g., Duchene Muscular Dystrophy (DMD))). SOMAscan, described in detail herein, has identified multiple protein drug targets. As described in the experimental section below, patients with DMD were analyzed using the compositions and methods of the invention resulting in the identification of protein drug targets for DMD. Experiments were performed that characterized protein expression of about 5% of the total proteome (e.g., just over about one thousand proteins) and identified several targets for therapeutic treatment (e.g., drug targets). As non-limiting examples, growth differentiation factor 11 (GDF-11), Receptor Expressed in Lymphoid Tissues (RELT), Complement decay-accelerating factor (CD55), gelsolin, and WFIKKN1 were identified.

As described in the Experimental section below, SOMAscan-measured proteins were analyzed and total amount of protein (as RFUs) in blood plotted as a function of the age of the DMD patients. In one embodiment, age is used as a surrogate for disease severity. In other embodiments, protein presence and expression in DMD patients is tested longitudinally.

Many proteins were elevated in the blood of young patients and showed diminishing concentrations as the patients aged. Most of these proteins appear to be intracellular muscle proteins whose concentrations in blood reflect muscle cell death. In the youngest patient these proteins are extremely high, as though muscle cell death may have been occurring embryonically.

GDF-11 displays a distinct age profile; at the earliest ages DMD patients have normal levels of GDF-11, with those levels falling over time in a manner consistent with low GDF-11 contributing to disease progression. Decreased levels of GDF-11 in blood are strongly correlated with diminished muscle function (both skeletal and cardiac muscle, which both fail in DMD patients over time). Injected GDF-11 corrects these phenotypes in mice. Since GDF-11 activity can be increased by direct injections of the purified GDF-11 protein or by direct injections of antagonists (e.g., monoclonal antibodies or SOMAmers) of natural inhibitors of GDF-11, the invention provides multiple options for treating and/or monitoring DMD patients (e.g., multiple methods of increasing expression in patients identified using compositions and methods of the invention that would benefit from increased expression of GDF-11).

Accordingly, in one embodiment, the invention provides methods for identifying a subject with muscular dystrophy (e.g., DMD) comprising detecting the level of one or more protein biomarkers (e.g., GDF-11, RELT, CD55, WFIKKN1, and/or gelsolin). In other embodiments, the invention provides methods of treating muscular dystrophy (e.g., DMD) in a subject via administering a therapeutically effective amount of one or more proteins identified utilizing the compositions and methods of the invention (e.g., aptamer based compositions and methods) as being a protein biomarker for DMD (e.g., administering one or more of GDF-11, RELT, CD55, WFIKKN1, and/or gelsolin to a subject with muscular dystrophy). In still further embodiments, the invention provides not only methods of treating (e.g., therapeutically or prophylactically), but also methods of monitoring the progression of muscular dystrophy (e.g., DMD), or monitoring the response to treatment, in a subject comprising detecting the levels of at least one, two or three protein biomarkers (e.g., GDF-11, RELT, CD55, WFIKKN1, and/or gelsolin) utilizing the aptamer based compositions and methods of the invention.

Embodiments of the present disclosure provide methods for detecting protein levels in biological samples. The present disclosure is illustrated with aptamer detection technology. However, the present disclosure is not limited to aptamer detection technology. Any suitable detection method (e.g., immunoassay, mass spectrometry, histological or cytological methods, etc.) is suitable for use herein.

In some embodiments, aptamer based assays involve the use of a microarray that includes one or more aptamers immobilized on a solid support. The aptamers are each capable of binding to a target molecule in a highly specific manner and with very high affinity. See, e.g., U.S. Pat. No. 5,475,096 entitled “Nucleic Acid Ligands”; see also, e.g., U.S. Pat. No. 6,242,246, U.S. Pat. No. 6,458,543, and U.S. Pat. No. 6,503,715, each of which is entitled “Nucleic Acid Ligand Diagnostic Biochip”. Once the microarray is contacted with a sample, the aptamers bind to their respective target molecules present in the sample and thereby enable a determination of a biomarker level corresponding to a biomarker.

Aptamers for use in the disclosure may include up to about 100 nucleotides, up to about 95 nucleotides, up to about 90 nucleotides, up to about 85 nucleotides, up to about 80 nucleotides, up to about 75 nucleotides, up to about 70 nucleotides, up to about 65 nucleotides, up to about 60 nucleotides, up to about 55 nucleotides, up to about 50 nucleotides, up to about 45 nucleotides, up to about 40 nucleotides, up to about 35 nucleotides, up to about 30 nucleotides, up to about 25 nucleotides, and up to about 20 nucleotides.

In another aspect of this disclosure, the aptamer has a dissociation constant (K_{d}) for its target of about 10 nM or less, about 15 nM or less, about 20 nM or less, about 25 nM or less, about 30 nM or less, about 35 nM or less, about 40 nM or less, about 45 nM or less, about 50 nM or less, or in a range of about 3-10 nM (or 3, 4, 5, 6, 7, 8, 9 or 10 nM.

An aptamer can be identified using any known method, including the SELEX process. Once identified, an aptamer can be prepared or synthesized in accordance with any known method, including chemical synthetic methods and enzymatic synthetic methods.

The terms “SELEX” and “SELEX process” are used interchangeably herein to refer generally to a combination of (1) the selection of aptamers that interact with a target molecule in a desirable manner, for example binding with high affinity to a protein, with (2) the amplification of those selected nucleic acids. The SELEX process can be used to identify aptamers with high affinity to a specific target or biomarker.

SELEX generally includes preparing a candidate mixture of nucleic acids, binding of the candidate mixture to the desired target molecule to form an affinity complex, separating the affinity complexes from the unbound candidate nucleic acids, separating and isolating the nucleic acid from the affinity complex, purifying the nucleic acid, and identifying a specific aptamer sequence. The process may include multiple rounds to further refine the affinity of the selected aptamer. The process can include amplification steps at one or more points in the process. See, e.g., U.S. Pat. No. 5,475,096, entitled “Nucleic Acid Ligands”. The SELEX process can be used to generate an aptamer that covalently binds its target as well as an aptamer that non-covalently binds its target. See, e.g., U.S. Pat. No. 5,705,337 entitled “Systematic Evolution of Nucleic Acid Ligands by Exponential Enrichment: Chemi-SELEX.”

The SELEX process can be used to identify high-affinity aptamers containing modified nucleotides that confer improved characteristics on the aptamer, such as, for example, improved in vivo stability or improved delivery characteristics. Examples of such modifications include chemical substitutions at the ribose and/or phosphate and/or base positions. SELEX process-identified aptamers containing modified nucleotides are described in U.S. Pat. No. 5,660,985, entitled “High Affinity Nucleic Acid Ligands Containing Modified Nucleotides”, which describes oligonucleotides containing nucleotide derivatives chemically modified at the 5′- and 2′-positions of pyrimidines. U.S. Pat. No. 5,580,737, see supra, describes highly specific aptamers containing one or more nucleotides modified with 2′-amino (2′-NH2), 2′-fluoro (2′-F), and/or 2′-O-methyl (2′-OMe). See also, U.S. Patent Application Publication No. 2009/0098549, entitled “SELEX and PHOTOSELEX”, which describes nucleic acid libraries having expanded physical and chemical properties and their use in SELEX and photoSELEX.

SELEX can also be used to identify aptamers that have desirable off-rate characteristics. See U.S. Publication No. US 2009/0004667, entitled “Method for Generating Aptamers with Improved Off-Rates”, which describes improved SELEX methods for generating aptamers that can bind to target molecules. Methods for producing aptamers and photoaptamers having slower rates of dissociation from their respective target molecules are described. The methods involve contacting the candidate mixture with the target molecule, allowing the formation of nucleic acid-target complexes to occur, and performing a slow off-rate enrichment process wherein nucleic acid-target complexes with fast dissociation rates will dissociate and not reform, while complexes with slow dissociation rates will remain intact. Additionally, the methods include the use of modified nucleotides in the production of candidate nucleic acid mixtures to generate aptamers with improved off-rate performance. In some embodiments, an aptamer comprises at least one nucleotide with a modification, such as a base modification. In some embodiments, an aptamer comprises at least one nucleotide with a hydrophobic modification, such as a hydrophobic base modification, allowing for hydrophobic contacts with a target protein. Such hydrophobic contacts, in some embodiments, contribute to greater affinity and/or slower off-rate binding by the aptamer.

In some embodiments, an aptamer comprises at least two, at least three, at least four, at least five, at least six, at least seven, at least eight, at least nine, or at least 10 nucleotides with hydrophobic modifications, where each hydrophobic modification may be the same or different from the others.

In some embodiments, a slow off-rate aptamer (including an aptamers comprising at least one nucleotide with a hydrophobic modification) has an off-rate (t_{1/2}) of ≧30 minutes, ≧60 minutes, ≧90 minutes, ≧120 minutes, ≧150 minutes, ≧180 minutes, ≧210 minutes, or ≧240 minutes.

In some embodiments, an assay employs aptamers that include photoreactive functional groups that enable the aptamers to covalently bind or “photocrosslink” their target molecules. See, e.g., U.S. Pat. No. 6,544,776 entitled “Nucleic Acid Ligand Diagnostic Biochip”. These photoreactive aptamers are also referred to as photoaptamers. See, e.g., U.S. Pat. No. 5,763,177, U.S. Pat. No. 6,001,577, and U.S. Pat. No. 6,291,184, each of which is entitled “Systematic Evolution of Nucleic Acid Ligands by Exponential Enrichment: Photoselection of Nucleic Acid Ligands and Solution SELEX”; see also, e.g., U.S. Pat. No. 6,458,539, entitled “Photoselection of Nucleic Acid Ligands”. After the microarray is contacted with the sample and the photoaptamers have had an opportunity to bind to their target molecules, the photoaptamers are photoactivated, and the solid support is washed to remove any non-specifically bound molecules. Harsh wash conditions may be used, since target molecules that are bound to the photoaptamers are generally not removed, due to the covalent bonds created by the photoactivated functional group(s) on the photoaptamers. In this manner, the assay enables the detection of a biomarker level corresponding to a biomarker in the test sample.

In some assay formats, the aptamers are immobilized on the solid support prior to being contacted with the sample. Under certain circumstances, however, immobilization of the aptamers prior to contact with the sample may not provide an optimal assay. For example, pre-immobilization of the aptamers may result in inefficient mixing of the aptamers with the target molecules on the surface of the solid support, perhaps leading to lengthy reaction times and, therefore, extended incubation periods to permit efficient binding of the aptamers to their target molecules. Further, when photoaptamers are employed in the assay and depending upon the material utilized as a solid support, the solid support may tend to scatter or absorb the light used to effect the formation of covalent bonds between the photoaptamers and their target molecules. Moreover, depending upon the method employed, detection of target molecules bound to their aptamers can be subject to imprecision, since the surface of the solid support may also be exposed to and affected by any labeling agents that are used. Finally, immobilization of the aptamers on the solid support generally involves an aptamer-preparation step (i.e., the immobilization) prior to exposure of the aptamers to the sample, and this preparation step may affect the activity or functionality of the aptamers.

Aptamer assays or “aptamer based assay(s)” that permit an aptamer to capture its target in solution and then employ separation steps that are designed to remove specific components of the aptamer-target mixture prior to detection have also been described (see U.S. Publication No. 2009/0042206, entitled “Multiplexed Analyses of Test Samples”). The described aptamer assay methods enable the detection and quantification of a non-nucleic acid target (e.g., a protein target) in a test sample by detecting and quantifying a nucleic acid (i.e., an aptamer). The described methods create a nucleic acid surrogate (i.e, the aptamer) for detecting and quantifying a non-nucleic acid target, thus allowing the wide variety of nucleic acid technologies, including amplification, to be applied to a broader range of desired targets, including protein targets.

Aptamers can be constructed to facilitate the separation of the assay components from an aptamer biomarker complex (or photoaptamer biomarker covalent complex) and permit isolation of the aptamer for detection and/or quantification. In one embodiment, these constructs can include a cleavable or releasable element within the aptamer sequence. In other embodiments, additional functionality can be introduced into the aptamer, for example, a labeled or detectable component, a spacer component, or a specific binding tag or immobilization element. For example, the aptamer can include a tag connected to the aptamer via a cleavable moiety, a label, a spacer component separating the label, and the cleavable moiety. In one embodiment, a cleavable element is a photocleavable linker. The photocleavable linker can be attached to a biotin moiety and a spacer section, can include an NHS group for derivatization of amines, and can be used to introduce a biotin group to an aptamer, thereby allowing for the release of the aptamer later in an assay method.

Homogenous assays, done with all assay components in solution, do not require separation of sample and reagents prior to the detection of signal. These methods are rapid and easy to use. These methods generate signal based on a molecular capture or binding reagent that reacts with its specific target. In some embodiments of the methods described herein, the molecular capture reagents comprise an aptamer or an antibody or the like and the specific target may be a biomarker shown in Example 1.

In some embodiments, a method for signal generation takes advantage of anisotropy signal change due to the interaction of a fluorophore-labeled capture reagent with its specific biomarker target. When the labeled capture reacts with its target, the increased molecular weight causes the rotational motion of the fluorophore attached to the complex to become much slower changing the anisotropy value. By monitoring the anisotropy change, binding events may be used to quantitatively measure the biomarkers in solutions. Other methods include fluorescence polarization assays, molecular beacon methods, time resolved fluorescence quenching, chemiluminescence, fluorescence resonance energy transfer, and the like.

An exemplary solution-based aptamer assay that can be used to detect a biomarker level in a biological sample includes the following: (a) preparing a mixture by contacting the biological sample with an aptamer that includes a first tag and has a specific affinity for the biomarker, wherein an aptamer affinity complex is formed when the biomarker is present in the sample; (b) exposing the mixture to a first solid support including a first capture element, and allowing the first tag to associate with the first capture element; (c) removing any components of the mixture not associated with the first solid support; (d) attaching a second tag to the biomarker component of the aptamer affinity complex; (e) releasing the aptamer affinity complex from the first solid support; (0 exposing the released aptamer affinity complex to a second solid support that includes a second capture element and allowing the second tag to associate with the second capture element; (g) removing any non-complexed aptamer from the mixture by partitioning the non-complexed aptamer from the aptamer affinity complex; (h) eluting the aptamer from the solid support; and (i) detecting the biomarker by detecting the aptamer component of the aptamer affinity complex. For example, protein concentration or levels in a sample may be expressed as relative fluorescence units (RFU), which may be a product of detecting the aptamer component of the aptamer affinity complex (e.g., aptamer complexed to target protein create the aptamer affinity complex). That is, for an aptamer-based assay, the protein concentration or level correlates with the RFU.

A nonlimiting exemplary method of detecting biomarkers in a biological sample using aptamers is described in Kraemer et al., PLoS One 6(10): e26332.

Aptamers may contain modified nucleotides that improve it properties and characteristics. Non-limiting examples of such improvements include, in vivo stability, stability against degradation, binding affinity for its target, and/or improved delivery characteristics.

Examples of such modifications include chemical substitutions at the ribose and/or phosphate and/or base positions of a nucleotide. SELEX process-identified aptamers containing modified nucleotides are described in U.S. Pat. No. 5,660,985, entitled “High Affinity Nucleic Acid Ligands Containing Modified Nucleotides,” which describes oligonucleotides containing nucleotide derivatives chemically modified at the 5′- and 2′-positions of pyrimidines. U.S. Pat. No. 5,580,737, see supra, describes highly specific aptamers containing one or more nucleotides modified with 2′-amino (2′-NH₂), 2′-fluoro (2′-F), and/or 2′-O-methyl (2′-OMe). See also, U.S. Patent Application Publication No. 20090098549, entitled “SELEX and PHOTOSELEX,” which describes nucleic acid libraries having expanded physical and chemical properties and their use in SELEX and photoSELEX.

Specific examples of a C-5 modification include substitution of deoxyuridine at the C-5 position with a substituent independently selected from: benzylcarboxyamide (alternatively benzylaminocarbonyl) (Bn), naphthylmethylcarboxyamide (alternatively naphthylmethylaminocarbonyl) (Nap), tryptaminocarboxyamide (alternatively tryptaminocarbonyl) (Trp), and isobutylcarboxyamide (alternatively isobutylaminocarbonyl) (iBu) as illustrated immediately below.

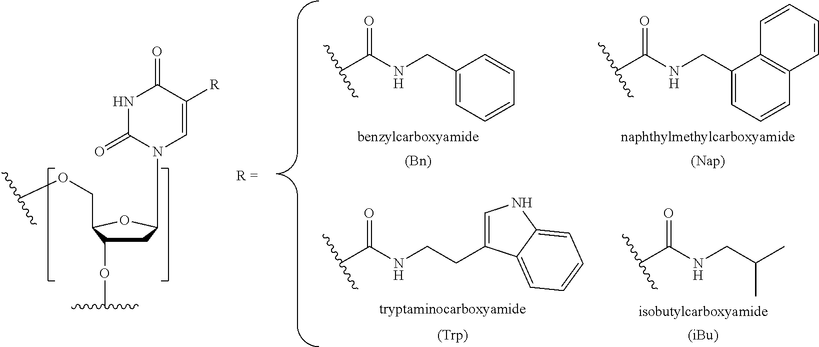

Chemical modifications of a C-5 modified pyrimidine can also be combined with, singly or in any combination, 2′-position sugar modifications, modifications at exocyclic amines, and substitution of 4-thiouridine and the like.

Representative C-5 modified pyrimidines include: 5-(N-benzylcarboxyamide)-2′-deoxyuridine (BndU), 5-(N-benzylcarboxyamide)-2′-O-methyluridine, 5-(N-benzylcarboxyamide)-2′-fluorouridine, 5-(N-isobutylcarboxyamide)-2′-deoxyuridine (iBudU), 5-(N-isobutylcarboxyamide)-2′-O-methyluridine, 5-(N-isobutylcarboxyamide)-2′-fluorouridine, 5-(N-tryptaminocarboxyamide)-2′-deoxyuridine (TrpdU), 5-(N-tryptaminocarboxyamide)-2′-O-methyluridine, 5-(N-tryptaminocarboxyamide)-2′-fluorouridine, 5-(N-[1-(3-trimethylamonium) propyl]carboxyamide)-2′-deoxyuridine chloride, 5-(N-naphthylmethylcarboxyamide)-2′-deoxyuridine (NapdU), 5-(N-naphthylmethylcarboxyamide)-2′-O-methyluridine, 5-(N-naphthylmethylcarboxyamide)-2′-fluorouridine or 5-(N-[1-(2,3-dihydroxypropyl)]carboxyamide)-2′-deoxyuridine).

If present, a modification to the nucleotide structure can be imparted before or after assembly of the polynucleotide. A sequence of nucleotides can be interrupted by non-nucleotide components. A polynucleotide can be further modified after polymerization, such as by conjugation with a labeling component.

Additional non-limiting examples of modified nucleotides (e.g., C-5 modified pyrimidine) that may be incorporated into the nucleic acid sequences of the present disclosure include the following:

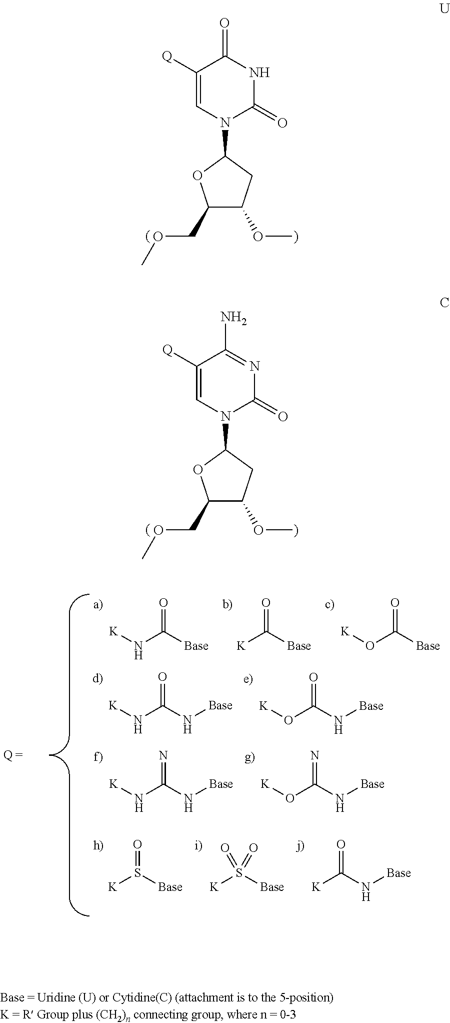

R′ is defined as follows:

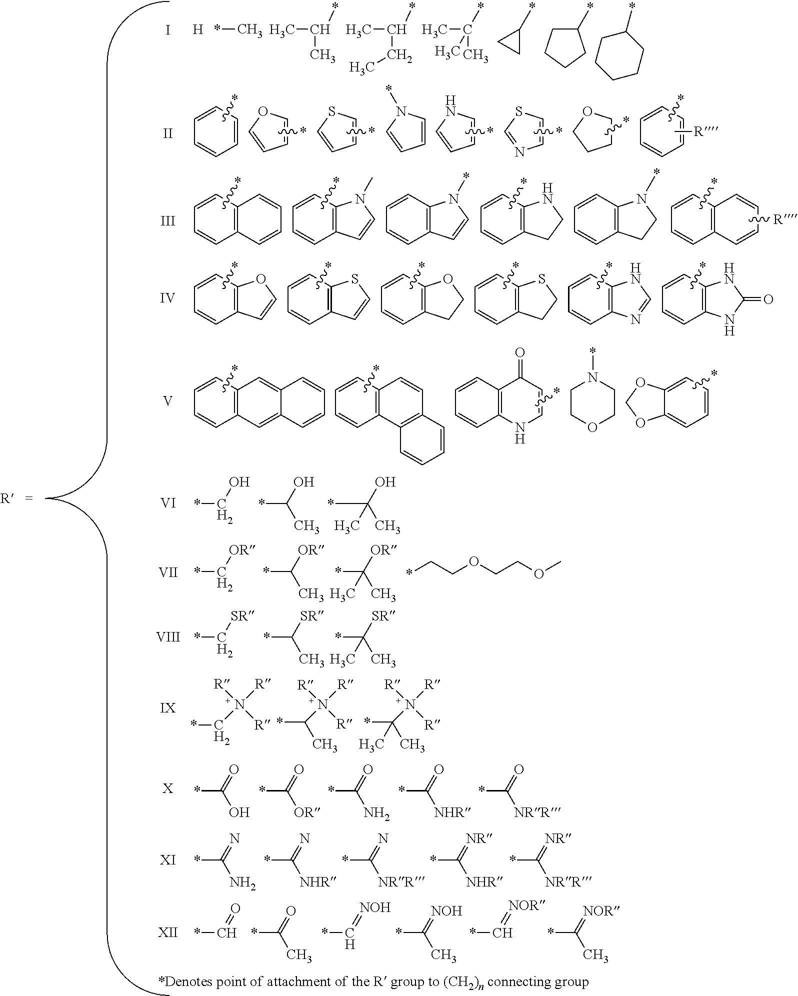

And, R″, R′″ and R″″ are defined as follows:
wherein
R′″ is selected from the group consisting of a branched or linear lower alkyl (C1-C20); halogen (F, Cl, Br, I); nitrile (CN); boronic acid (BO₂H₂); carboxylic acid (COOH); carboxylic acid ester (COOR″); primary amide (CONH₂); secondary amide (CONHR″); tertiary amide (CONR″R′″); sulfonamide (SO₂NH₂); N-alkylsulfonamide (SONHR″).
wherein
R″, R′″ are independently selected from a group consisting of a branched or linear lower alkyl (C1-C2)); phenyl (C₆H₅); an R″′ substituted phenyl ring (R″″C₆H₄); wherein R″″ is defined above; a carboxylic acid (COOH); a carboxylic acid ester (COOR′″″); wherein R′″″ is a branched or linear lower alkyl (C1-C20); and cycloalkyl; wherein R″═R′″═(CH₂); wherein n=2-10.

Further, C-5 modified pyrimidine nucleotides include the following:

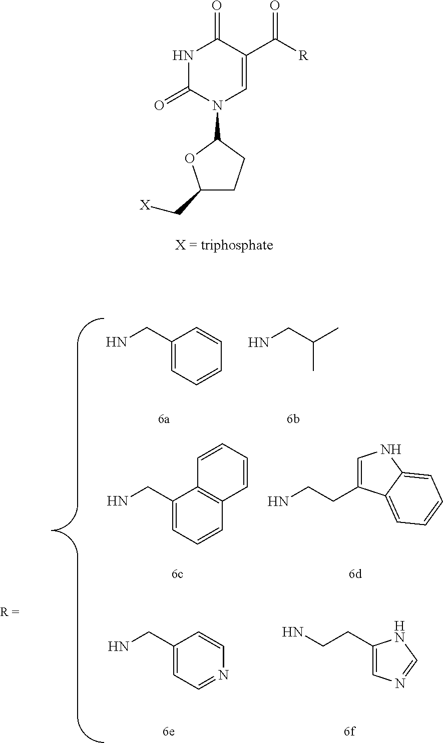

In some embodiments, the modified nucleotide confers nuclease resistance to the oligonucleotide. A pyrimidine with a substitution at the C-5 position is an example of a modified nucleotide. Modifications can include backbone modifications, methylations, unusual base-pairing combinations such as the isobases isocytidine and isoguanidine, and the like. Modifications can also include 3′ and 5′ modifications, such as capping. Other modifications can include substitution of one or more of the naturally occurring nucleotides with an analog, internucleotide modifications such as, for example, those with uncharged linkages (e.g., methyl phosphonates, phosphotriesters, phosphoamidates, carbamates, etc.) and those with charged linkages (e.g., phosphorothioates, phosphorodithioates, etc.), those with intercalators (e.g., acridine, psoralen, etc.), those containing chelators (e.g., metals, radioactive metals, boron, oxidative metals, etc.), those containing alkylators, and those with modified linkages (e.g., alpha anomeric nucleic acids, etc.). Further, any of the hydroxyl groups ordinarily present on the sugar of a nucleotide may be replaced by a phosphonate group or a phosphate group; protected by standard protecting groups; or activated to prepare additional linkages to additional nucleotides or to a solid support. The 5′ and 3′ terminal OH groups can be phosphorylated or substituted with amines, organic capping group moieties of from about 1 to about 20 carbon atoms, polyethylene glycol (PEG) polymers in one embodiment ranging from about 10 to about 80 kDa, PEG polymers in another embodiment ranging from about 20 to about 60 kDa, or other hydrophilic or hydrophobic biological or synthetic polymers. In one embodiment, modifications are of the C-5 position of pyrimidines. These modifications can be produced through an amide linkage directly at the C-5 position or by other types of linkages.

Polynucleotides can also contain analogous forms of ribose or deoxyribose sugars that are generally known in the art, including 2′-O-methyl-, 2′-O-allyl, 2′-fluoro- or 2′-azido-ribose, carbocyclic sugar analogs, a-anomeric sugars, epimeric sugars such as arabinose, xyloses or lyxoses, pyranose sugars, furanose sugars, sedoheptuloses, acyclic analogs and abasic nucleoside analogs such as methyl riboside. As noted above, one or more phosphodiester linkages may be replaced by alternative linking groups. These alternative linking groups include embodiments wherein phosphate is replaced by P(0)S (“thioate”), P(S)S (“dithioate”), (0)NR₂ (“amidate”), P(O)R, P(0)OR′, CO or CH₂ (“formacetal”), in which each R or R′ is independently H or substituted or unsubstituted alkyl (1-20 C) optionally containing an ether (-0-) linkage, aryl, alkenyl, cycloalky, cycloalkenyl or araldyl. Not all linkages in a polynucleotide need be identical. Substitution of analogous forms of sugars, purines, and pyrimidines can be advantageous in designing a final product, as can alternative backbone structures like a polyamide backbone, for example.

The present disclosure provides kits comprising aptamers described herein. Such kits can comprise, for example, (1) at least one aptamer for identification of a protein target; and (2) at least one pharmaceutically acceptable carrier, such as a solvent or solution. Additional kit components can optionally include, for example: (1) any of the pharmaceutically acceptable excipients identified herein, such as stabilizers, buffers, etc., (2) at least one container, vial or similar apparatus for holding and/or mixing the kit components; and (3) delivery apparatus.

In some embodiments, the present disclosure provides systems and methods for identifying proteins with altered expression in subjects with disease relative to subjects that do not have the disease. In some embodiments, proteins with altered expression serve as targets for drug screening and therapeutic applications. For example, in some embodiments, customized treatment is provided that is individualized to the proteomic profile of an individual subject's disease.

In some embodiments, proteins with altered expression are identified as targets for drug discovery. In some embodiments, proteins with existing drugs that target them are identified and such drugs are administered (alone or in combination with other drugs) to a subject. Thus, in some embodiments, the present disclosure provides customized treatment for a disease or condition.

In some embodiments, protein expression is compared to a reference sample from a disease-free subject or population of subjects. In some embodiments, the reference sample is sample of normal tissue from the subject, or a population average of normal tissue. In some embodiments, the level of the proteins is altered at least 2-fold (e.g., at least 4-fold, at least 5-fold, at least 10-fold, at least 20-fold, at least 50-fold, at least 100-fold, or more). The present disclosure is suitable for identification of altered protein expression (e.g., using the assays described herein) in a variety of sample types. Examples include, but are not limited to, tissue, whole blood, leukocytes, peripheral blood mononuclear cells, buffy coat, plasma, serum, sputum, tears, mucus, nasal washes, nasal aspirate, breath, urine, semen, saliva, peritoneal washings, ascites, cystic fluid, meningeal fluid, amniotic fluid, glandular fluid, pancreatic fluid, lymph fluid, pleural fluid, cytologic fluid, nipple aspirate, bronchial aspirate, bronchial brushing, synovial fluid, joint aspirate, organ secretions, cells, a cellular extract, or cerebrospinal fluid.

The present disclosure is not limited to the identification of targets for a particular disease. In some embodiments, the disease is, for example, a muscular disease (e.g., DMD), a genetic disease, a metabolic disorder, an inflammatory disease, or an infectious disease. In some embodiments, the disease is DMD and the drug targets are one or more of GDF-11, RELT, CD55, and/or other protein identified utilizing the compositions and methods of the invention.

In some embodiments, a computer-based analysis program is used to translate the raw data generated by the detection assay (e.g., the presence, absence, or amount of a given marker or markers) into data of value for a clinician (e.g., drug targets or drug(s) selection). The clinician can access the data using any suitable means. Thus, in some preferred embodiments, the present invention provides the further benefit that the clinician, who is not likely to be trained in genetics or molecular biology, need not understand the raw data. The data is presented directly to the clinician in its most useful form. The clinician is then able to immediately utilize the information in order to optimize the care of the subject.

The present invention contemplates any method capable of receiving, processing, and transmitting the information to and from laboratories conducting the assays, information providers, medical personal, and subjects. For example, in some embodiments of the present invention, a sample (e.g., a biopsy or other sample) is obtained from a subject and submitted to a profiling service (e.g., clinical lab at a medical facility, genomic profiling business, etc.), located in any part of the world (e.g., in a country different than the country where the subject resides or where the information is ultimately used) to generate raw data. Where the sample comprises a tissue or other biological sample, the subject may visit a medical center to have the sample obtained and sent to the profiling center, or subjects may collect the sample themselves (e.g., a urine sample) and directly send it to a profiling center. Where the sample comprises previously determined biological information, the information may be directly sent to the profiling service by the subject (e.g., an information card containing the information may be scanned by a computer and the data transmitted to a computer of the profiling center using an electronic communication systems). Once received by the profiling service, the sample is processed and a profile is produced (e.g., protein expression data), specific for the diagnostic, therapeutic, or prognostic information desired for the subject.

The profile data is then prepared in a format suitable for interpretation by a treating clinician. For example, rather than providing raw expression data, the prepared format may represent a suggested treatment course of action (e.g., specific drugs for administration). The data may be displayed to the clinician by any suitable method. For example, in some embodiments, the profiling service generates a report that can be printed for the clinician (e.g., at the point of care) or displayed to the clinician on a computer monitor.

In some embodiments, the information is first analyzed at the point of care or at a regional facility. The raw data is then sent to a central processing facility for further analysis and/or to convert the raw data to information useful for a clinician or patient. The central processing facility provides the advantage of privacy (all data is stored in a central facility with uniform security protocols), speed, and uniformity of data analysis. The central processing facility can then control the fate of the data following treatment of the subject. For example, using an electronic communication system, the central facility can provide data to the clinician, the subject, or researchers.

In some embodiments, the subject is able to directly access the data using the electronic communication system. The subject may chose further intervention or counseling based on the results. In some embodiments, the data is used for research use. For example, the data may be used to further optimize the inclusion or elimination of markers as useful indicators of a treatment outcome or for drug discovery.

### EXAMPLES

The following examples are provided to illustrate certain particular features and/or embodiments. These examples should not be construed to limit the disclosure to the particular features or embodiments described.

### Example 1

### Identification and Characterization of Protein Biomarkers in Duchenne Muscular Dystrophy

The modified-aptamer based screening technology (the SOMAscan assay, described in detail herein) was used for biomarker discovery and validation. In particular, the SOMAscan technology was used to screen for biomarkers associated with Duchenne muscular dystrophy (DMD) using serum samples from two independent cohorts collected in different geographies and run at different times. The first cohort analyzed was from The Parent Project Muscular Dystrophy-Cincinnati Children's Hospital Medical Center (PPMD-C), which included the goal of identifying a non-dystrophin-centric path to treatment for patients with DMD. The second cohort analyzed was from The Cooperative International Neuromuscular Research Group (CINRG, headquartered at Children's National Medical Center in Washington, D.C.), which included the goal of identifying changes in biomarkers with age in patients with DMD. In the current study, the data from these two independent studies were compared. This enabled identification of 44 concordant biomarkers in the blood associated with DMD, including 24 that are significantly increased and 20 that are significantly decreased in patients with DMD. As described herein, the invention provides new diagnostic, prognostic and therapeutic approaches for disease management.

Two independent DMD natural history cohorts were used in this study. The Parent Project Muscular Dystrophy-Cincinnati Children's Hospital Medical Center (PPMD-C) cohort comprised 42 DMD patients (2 to 27 years old) and 28 healthy volunteers (4 to 28 years old—most often from the DMD male sibling pool). The Cooperative International Neuromuscular Research Group (CINRG) cohort comprised 51 DMD patients (age range 4 to 29 years old) and 17 healthy volunteers (age range, 6 to 18 years old). The demographics and characteristics of the two cohorts are summarized in Table 1. The PPMD-C study included steroid treatment as a variable in the initial analysis, and the CINRG study included ambulatory status. Steroid treatment had no statistically significant effect on the 44 protein biomarkers described below, and ambulatory status was relevant only insofar as it related to increasing age but had no statistically significant effect on the results. Our standard quality control protocols detected no significant difference in the samples from the two cohorts.

PPMD-C: After obtaining informed consent, at least 10004 of serum was collected from each member of this cohort through Cincinnati Children's Hospital Medical Center using collection protocols and kits supplied by SomaLogic, including shipping to SomaLogic on dry ice shortly after collection.

CINRG cohort: Sera samples from DMD patients (n=51) and age matched healthy volunteers (n=17) were collected through the Cooperative International Neuromuscular Research Group (CINRG) network according to an approved institutional IRB protocol and used for an independent biomarker discovery experiment for human subjects. Sera samples were gathered mostly from two CINRG sites; UC Davis and Alberta Children's Hospital sites. These samples were collected over span of 3-6 months and stored in 1004 aliquots at −80° C. at Children's National Medical Center (Washington D.C.) for another 3 months before being sent to SomaLogic for analysis.

TABLE 1
Demographics and characteristics of the PPMD-C and CINRG cohorts.

| PPMD-C | | | CINRG | | | |
|---|---|---|---|---|---|---|
| Sampleld | AGE | DIAGNOSIS | Sampleld | AGE | DIAGNOSIS | Ambulation |
| 101 | 8 | DMD_S | 6 | 14.9 | DMD | NO |
| 102 | 13 | DMD_S | 30 | 26.6 | DMD | NO |
| 103 | 7 | DMD_S | 31 | 11.7 | DMD | NO |
| 104 | 10 | DMD_S | 45 | 24.4 | DMD | NO |
| 105 | 19 | DMD_S | 46 | 27.4 | DMD | NO |
| 106 | 11 | DMD_S | 56 | 15.9 | DMD | NO |
| 107 | 11 | DMD_S | 63 | 28.7 | DMD | NO |
| 108 | 16 | DMD_S | 94 | 27.2 | DMD | NO |
| 110 | 10 | DMD_S | 97 | 13.6 | DMD | YES |
| 111 | 8 | DMD_S | 115 | 15 | DMD | YES |
| 112 | 5 | DMD_S | 145 | 18.7 | DMD | NO |
| 113 | 14 | DMD_S | 149 | 14.5 | DMD | NO |
| 114 | 9 | DMD_S | 150 | 19.9 | DMD | NO |
| 115 | 12 | DMD_S | 152 | 11.4 | DMD | NO |
| 116 | 7 | DMD_S | 167 | 17.8 | DMD | YES |
| 117 | 14 | DMD_S | 168 | 11.2 | DMD | YES |
| 118 | 10 | DMD_S | 218 | 15 | DMD | NO |
| 119 | 9 | DMD_S | 230 | 15.9 | DMD | NO |
| 120 | 7 | DMD_S | 231 | 11.7 | DMD | NO |
| 121 | 14 | DMD_S | 252 | 25.2 | DMD | NO |
| 122 | 4 | DMD_S | 268 | 20.6 | DMD | NO |
| 123 | 11 | DMD_S | 317 | 17.2 | DMD | NO |
| 125 | 9 | DMD_S | 319 | 14.3 | DMD | NO |
| 126 | 10 | DMD_S | 339 | 9.6 | DMD | YES |
| 127 | 8 | DMD_S | 356 | 9.2 | DMD | YES |
| 128 | 10 | DMD_S | 357 | 16.8 | DMD | NO |
| 129 | 8 | DMD_S | 360 | 14.9 | DMD | YES |
| 130 | 12 | DMD_S | 369 | 23.6 | DMD | NO |
| 201 | 20 | DMD | 381 | 9.5 | DMD | YES |
| 202 | 5 | DMD | 382 | 10.8 | DMD | YES |
| 203 | 18 | DMD | 391 | 26.8 | DMD | NO |
| 204 | 20 | DMD | 392 | 6.8 | DMD | YES |
| 205 | 21 | DMD | 410 | 12.1 | DMD | YES |
| 206 | 19 | DMD | 80301 | 5.6 | DMD | YES |
| 207 | 13 | DMD | 80302 | 4.3 | DMD | YES |
| 208 | 15 | DMD | 81901 | 5.6 | DMD | YES |
| 209 | 2 | DMD | 81902 | 7.9 | DMD | YES |
| 210 | 16 | DMD | 81903 | 4.4 | DMD | YES |
| 211 | 20 | DMD | 81904 | 6.3 | DMD | YES |
| 212 | 12 | DMD | 81905 | 4.4 | DMD | YES |
| 214 | 7 | DMD | 81906 | 4.9 | DMD | YES |
| 215 | 15 | DMD | 81907 | 7.4 | DMD | YES |
| 301 | 20 | CONTROL | 81908 | 5.7 | DMD | YES |
| 302 | 9 | CONTROL | 82102 | 8 | DMD | YES |
| 303 | 17 | CONTROL | 82301 | 4.3 | DMD | YES |
| 304 | 19 | CONTROL | 82302 | 5.4 | DMD | YES |
| 305 | 13 | CONTROL | 82303 | 5 | DMD | YES |
| 306 | 16 | CONTROL | 82304 | 5 | DMD | YES |
| 307 | 14 | CONTROL | 82305 | 4 | DMD | YES |
| 308 | 10 | CONTROL | 82306 | 4.3 | DMD | YES |
| 309 | 12 | CONTROL | 82307 | 7.7 | DMD | YES |
| 310 | 11 | CONTROL | 182301 | 15.6 | CONTROL | |
| 311 | 14 | CONTROL | 182303 | 15 | CONTROL | |
| 312 | 9 | CONTROL | 182304 | 13.3 | CONTROL | |
| 313 | 11 | CONTROL | 182306 | 13.7 | CONTROL | |
| 314 | 27 | CONTROL | 182308 | 15.8 | CONTROL | |
| 315 | 14 | CONTROL | 182309 | 17.3 | CONTROL | |
| 316 | 12 | CONTROL | 182312 | 13 | CONTROL | |
| 317 | 15 | CONTROL | 182313 | 14.5 | CONTROL | |
| 318 | 8 | CONTROL | 182318 | 17.4 | CONTROL | |
| 319 | 8 | CONTROL | 182319 | 11.6 | CONTROL | |
| 320 | 9 | CONTROL | 182322 | 6 | CONTROL | |
| 321 | 10 | CONTROL | 182323 | 10.3 | CONTROL | |
| 322 | 13 | CONTROL | 182324 | 8 | CONTROL | |
| 323 | 19 | CONTROL | 182325 | 13.6 | CONTROL | |
| 324 | 11 | CONTROL | 182327 | 8.4 | CONTROL | |
| 325 | 5 | CONTROL | 182328 | 12.3 | CONTROL | |
| 327 | 21 | CONTROL | | | | |
| 329 | 8 | CONTROL | | | | |
| 330 | 10 | CONTROL | | | | |

Serum samples were tested using SOMAscan protein biomarker discovery assay (SomaLogic, Inc.), which detects 1,125 proteins simultaneously using 65 microliters of serum. DMD and control samples were randomly assigned to plates within the each assay run along with a set of calibration and normalization samples. No identifying information was available to the laboratory technicians operating the assay. Intra-run normalization and inter-run calibration were performed according to SOMAscan Version 3 assay data quality control (QC) procedures as defined in the SomaLogic good laboratory practice (GLP) quality system. Samples from the PPMD-C and CINRG cohorts were assayed independently and data from all samples passed QC criteria and were fit for analysis.

SOMAscan proteomic data is reported in relative fluorescence units (RFU). RFU data were log transformed prior to statistical analysis to reduce heteroscedasticity. The non-parametric Kolmogorov-Smirnoff (KS) test was used to identify differentially expressed proteins between DMD and controls. The KS test statistic is an unsigned quantity—here we include a sign to indicate the direction of the differential expression with positive test statistics indicating higher signal levels in DMD patients than in controls. All statistical analysis performed with the R language for statistical computing version 3.1.2 (2014-10-31).

We report the false discovery rate (FDR) computed with the R package stats (R Core Team 2014. R: A language and environment for statistical computing. R Foundation for Statistical Computing, Vienna, Austria. R-project.org). Repeated application of a statistical test results in Type I error rates that exceed the nominal rate associated with a single test. This well-known phenomenon is typically addressed by applying a “multiple-testing” correction to the nominal significance level. For example, the Bonferroni correction is often used to control the “family-wise” error rate, i.e., the probability of generating at least one false positive result in a family of repeated hypothesis tests. A Bonferroni-corrected p-value is calculated by multiplying each p-value by the number of comparisons. In “large scale” hypothesis testing situations such as this study, controlling the probability of one or more false positives is often less informative than controlling the expected number of false positives. The latter has been dubbed the “false discovery rate” or FDR. FDR-adjusted p-values serve as a guide to assessing statistical significance by indicating the expected number of false discoveries at a given significance level, though even this technique has limitations that can appear in small studies.

At a stringent 1% false discovery rate-corrected significance level (see Materials and Methods), based on SOMAscan data from a total of 93 DMD patients and 45 age-matched controls from the two cohorts, 44 proteins were identified that consistently differed in the serum in both cohorts when comparing DMD patients vs. controls. The names and Kolmogorov-Smirnov (KS) distances for these 44 proteins in each cohort (and averaged between cohorts) are shown in Table 2, along with each protein's known enrichment in muscle tissue.

TABLE 2
Proteins present at altered levels in DMD patients

| | | PPMD-C | CINRG | | | | Age- |
|---|---|---|---|---|---|---|---|
| | Gene | signed | signed | | | | related |
| | name | KS | KS | Average | | Muscle | change |
| Protein name (UniProt) | (UniProt) | distance | distance | KS | Rank | enriched | group no. |
| Troponin I, fast skeletal muscle | TNNI2 | 1.000 | 0.918 | 0.959 | 1 | Yes | 1 |
| Carbonic anhydrase 3 | CA3 | 0.964 | 0.938 | 0.951 | 2 | Yes | 1 |
| Fatty acid-binding protein, heart | FABP3 | 1.000 | 0.882 | 0.941 | 3 | Yes | 1 |
| Troponin I, cardiac muscle | TNNI3 | 0.917 | 0.961 | 0.939 | 4 | Yes | 1 |
| Creatine kinase M-type | CKM | 0.976 | 0.839 | 0.908 | 5 | Yes | 1 |
| Mitogen-activated protein kinase 12 | MAPK12 | 1.000 | 0.797 | 0.898 | 6 | Yes | 1 |
| Alanine aminotransferase 1 | GPT | 0.738 | 0.941 | 0.840 | 7 | No | 1 |
| Myoglobin | MB | 0.857 | 3.824 | 0.838 | 8 | Yes | 1 |
| Fibrinogen | FGA FGR FGG | 0.810 | 0.784 | 0.797 | 9 | No | 1 |
| Phospholipase A2, | PLA2G2A | 0.762 | 0.800 | 0.781 | 10 | No | 3 |
| membrane associated | | | | | | | |
| Acidic leucine-rich nuclear | ANP32B | 0.821 | 0.706 | 0.764 | 11 | No | 1 |
| phosphoprotein 32 | | | | | | | |
| family member B | | | | | | | |
| Hepatoma-derived growth factor- | HDGFRP2 | 0.738 | 0.691 | 0.715 | 12 | No | 3 |
| related protein 2 | | | | | | | |
| 40S ribosomal protein S7 | RPS7 | 0.690 | 0.734 | 0.712 | 13 | No | 1 |
| Glucose-6-phosphate isomerase | GPI | 0.774 | 0.604 | 0.689 | 14 | Yes | 1 |
| Heparin cofactor 2 | SERPIND1 | 0.560 | 0.813 | 0.686 | 15 | No | 3 |
| Persephin | PSPN | 0.595 | 0.757 | 0.676 | 16 | No | 3 |
| Calcium/calmodulin-dependent | CAMK2A | 0.738 | 0.586 | 0.662 | 17 | Yes | 1 |
| protein kinase II α | | | | | | | |
| Malate dehydrogenase, cytoplasmic | MDH1 | 0.595 | 0.706 | 0.651 | 18 | Yes | 1 |
| c-lactate dehydrogenase B chain | LDHB | 0.631 | 0.608 | 0.619 | 19 | Yes | 1 |
| Aminoacylase-1 | ACY1 | 0.643 | 0.577 | 0.610 | 20 | No | 1 |
| Proteosome subunit α type-2 | PSMA2 | 0.571 | 0.600 | 0.586 | 21 | No | 3 |
| C-X-C motif chemokine 10 | CXCL10 | 0.560 | 0.600 | 0.580 | 22 | No | 3 |
| cAMP-dependent protein kinase | PRKACA | 0.560 | 0.570 | 0.565 | 23 | No | 1 |
| catalytic subunit α | | | | | | | |
| Heat-shock 70 kDa protein 1A/1B | HSPA1A | 0.476 | 0.600 | 0 538 | 24 | Yes | 1 |
| Proto-oncogene tyrosine-protein | RET | −0.917 | −0.961 | −0.939 | 1 | No | 2 |
| kinase receptor Ret | | | | | | | |
| Growth/differentiation factor 11 | GDF11 | −0.667 | −0.941 | −0.804 | 2 | No | 4 |
| Complement decay- | CD55 | −0.762 | −0.745 | −0.754 | 3 | No | 4 |
| accelerating factor | | | | | | | |
| Cadherin-5 | CDH5 | −0.821 | −0.675 | −0.748 | 4 | No | 2 |
| Tumor necrosis factor receptor | RELT | −0.786 | −0.706 | −0.746 | 5 | No | 4 |
| superfamily member 19L | | | | | | | |
| Gelsolin | GSN | −0.750 | −0.718 | −0.734 | 6 | Yes | 4 |
| Wnt inhibitory factor 1 | WIF1 | −0.679 | −0.714 | −0.697 | 7 | No | 2 |
| Contactin-5 | CNTN5 | −0.655 | −0.702 | −0.678 | 8 | No | 2 |
| Prolyl endopeptidase FAP | FAP | −0.643 | −0.659 | −0.651 | 9 | No | 2 |
| Jagged-1 | JAG1 | −0.679 | −0.613 | −0.646 | 10 | No | 2 |
| Netrin receptor UNC5C | UNC5C | −0.560 | −0.718 | −0.639 | 11 | No | 2 |
| Kunitz-type protease inhibitor 1 | SPINT1 | −0.667 | −0.597 | −0.632 | 12 | No | 2 |
| Protein SET | SET | −0.500 | −0.722 | −0.611 | 13 | No | 2 |
| Disintegrin & metalloproteinase | ADAM9 | −0.595 | −0.600 | −0.598 | 14 | No | 2 |
| domain-containing protein 9 | | | | | | | |
| Cell adhesion molecule L1-like | CHL1 | −0.583 | −0.589 | −0.586 | 15 | No | 2 |
| Osteomodulin | OMD | −0.452 | −0.718 | −0.585 | 16 | No | 2 |
| WAP, Kazal, Ig, Kunitz and NTR | WFIKKN1 | −0.464 | −0.699 | −0.581 | 17 | No | 4 |
| domain-containing protein 1 | | | | | | | |
| Bone sialoprotein 2 | IBSP | −0.476 | −0.613 | −0.544 | 18 | No | 2 |
| Interleukin-34 | IL34 | −0.488 | −0.558 | −0.523 | 19 | No | 2 |
| Neurogenic locus notch | NOTCH3 | −0.488 | −0.550 | −0.519 | 20 | No | 2 |
| homolog protein 3 | | | | | | | |

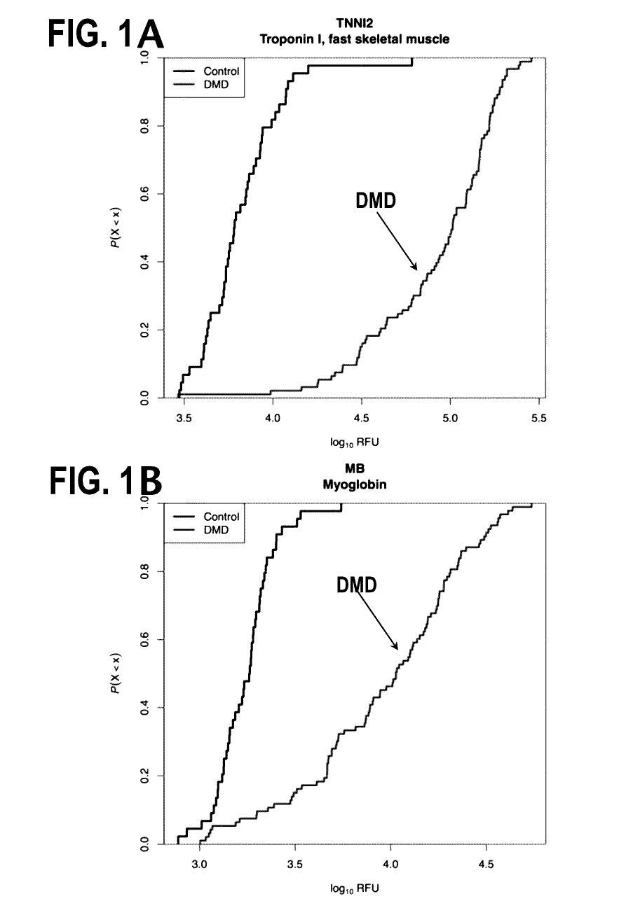

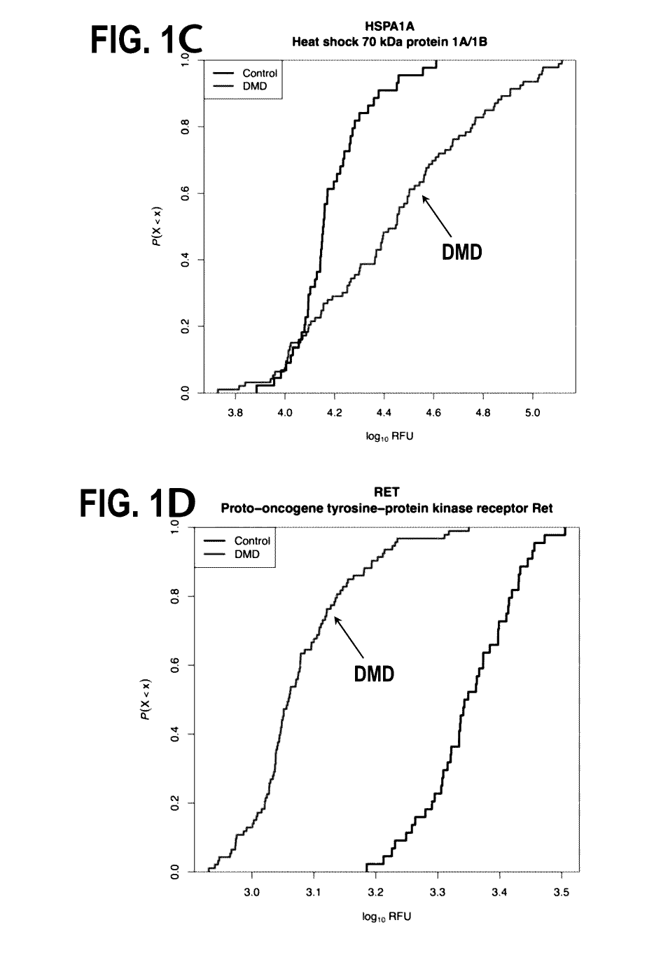

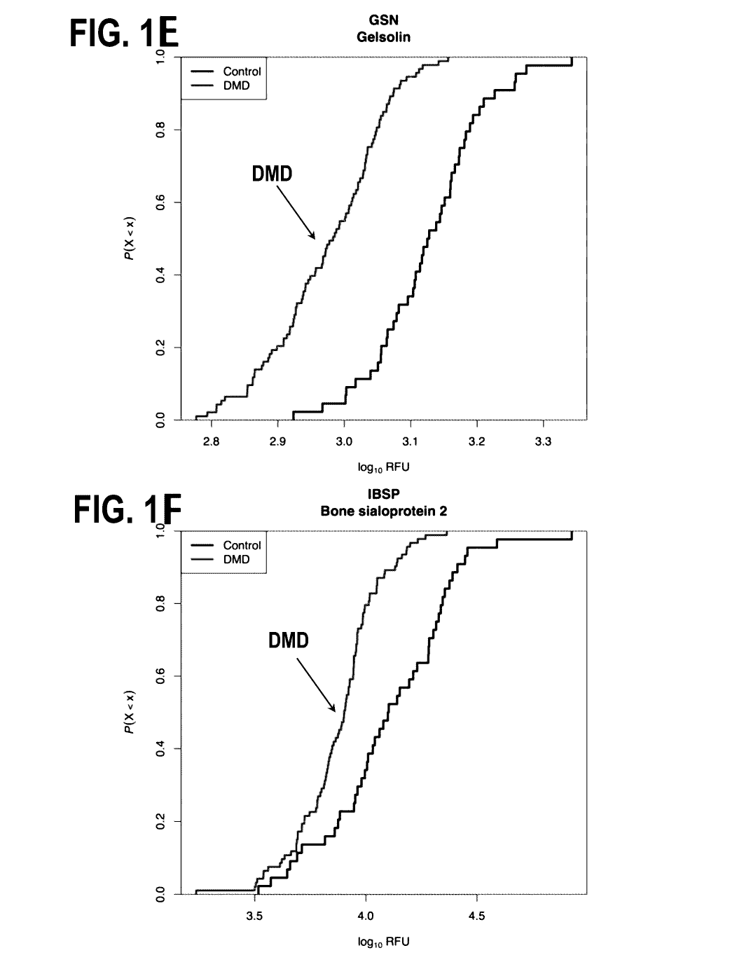

Of the 44 protein biomarkers that were significantly different between DMD and controls, 24 increased and 20 decreased in detection in DMD compared to normal controls. FIG. 1 shows empirical Cumulative Distribution Function (CDF) plots for six representative proteins from the combined cohort analysis (three proteins that are increased—troponin 1 fast skeletal muscle (TNNI2); myoglobin (MB); heat shock protein 70 (Hsp70, or HSPA1A)—and three that are decreased—proto-oncogene tyrosine-protein kinase receptor Ret (RET); gelsolin (GSN); bone sialoprotein 2 (IBSP)—in DMD patients vs. controls). These examples range from the highest KS distance (near 1 or −1) to the lowest significant (near 0.5 or −0.5) for both the “up” and “down” groups respectively.

Empirical Cumulative Distribution Function (CDF) plots for six representative proteins from the combined cohort analysis (three proteins that are increased in DMD patients vs. controls: troponin 1 fast skeletal muscle, myoglobin, heat shock protein 1; and three proteins that are decreased in DMD patients vs. controls: RET, gelsolin, bone sialoprotein 2) are shown in FIG. 1. These examples range from the highest KS distance (near 1) to the lowest significance (near 0.5) for both the “up” and “down” groups. For reference, some of the 44 proteins are present at levels that are as different in DMD patients from controls as is human chorionic gonadotropin (hCG) in pregnant vs. non-pregnant women.

### Example 2

### Correlation Between Biomarker Levels and Age of DMD Patients

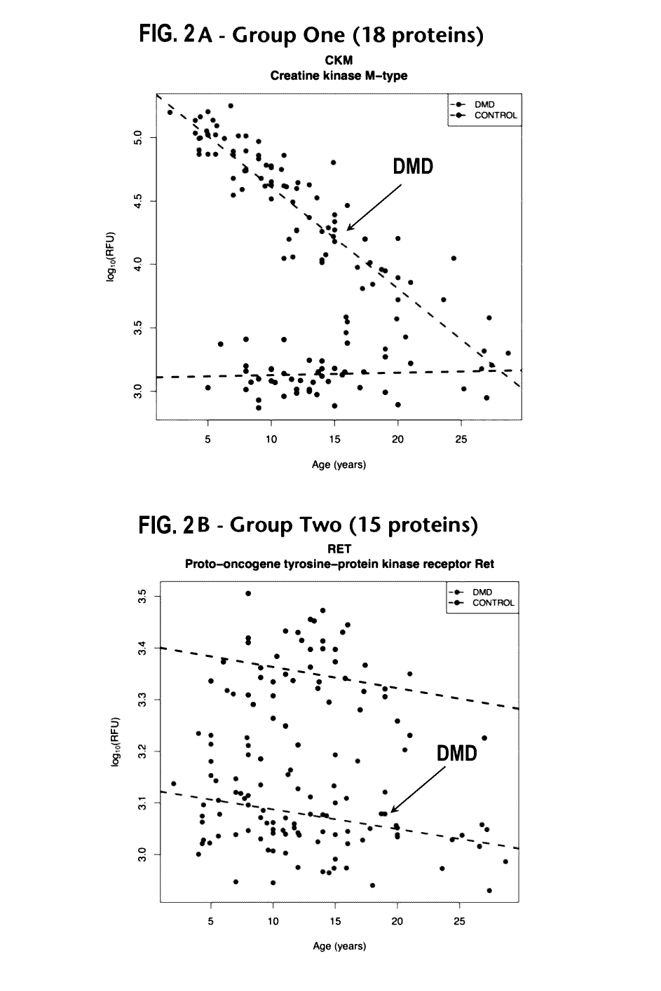

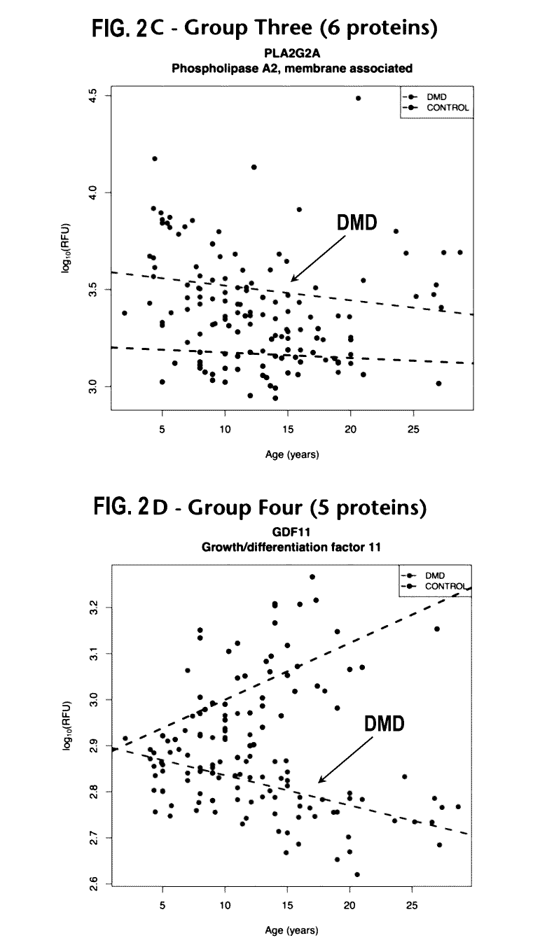

Age was a proxy for disease severity, as older patients have more advanced disease. Since multiple samples taken over time were not available for individual patients, the age-dependence in protein levels across the whole cohort was examined. Proteins were screened using a single protein linear regression model to identify candidates where patient age was a useful predictor of protein concentration. Four general groupings were identified for the differential age-related protein changes for the 44 biomarkers identified. See FIG. 2.
1) Protein biomarkers that were at their highest levels in young DMD patients—much higher than in normal controls—and then decrease as a function of age in DMD while remaining relatively unchanged with age in controls (18 proteins, represented by creatine kinase, FIG. 2A);
2) Proteins that changed with age in DMD patients and controls, but which were significantly lower in DMD patients through most age points (15 proteins, represented by RET, FIG. 2B);
3) Protein biomarkers that changed with age in DMD patients and controls, but which were significantly higher in DMD patients at most age points (6 proteins, represented by phospholipase A2, FIG. 2C), and
4) Protein biomarkers whose concentrations were similar between DMD patient and controls at an early age, but then decreased with age in DMD patients while increasing in controls (5 proteins, represented by growth differentiation factor 11 (GDF-11), FIG. 2D).

### Example 3

### Discussion

Using the SOMAscan assay, 44 circulating serum biomarkers associated with DMD patients vs. healthy controls were identified from two independent cohorts with a 1% false discovery rate-corrected significance level.

The greatest differences between DMD patients and controls were observed in the young age range (4 to 10 years old), when certain biomarkers were elevated up to two orders of magnitude in serum samples of DMD patients relative to healthy volunteers; these biomarkers then declined with age and ensuing disease progression. These “creatine kinase-like biomarkers” (FIG. 2A) are mostly of muscle origin and their elevation is likely associated with muscle damage/cell death and inflammation at an early age, and their subsequent decline with age may be the result of loss of muscle mass in the DMD patients.

The decrease in levels of these proteins may reflect myofiber membrane instability/damage, necrosis, and leakage of cytoplasm into the extracellular space. This group includes muscle-enriched proteins such as creatine kinase M-type (CK-M) itself, fatty acid binding protein 3 (FABP3), myoglobin (MB), carbonic anhydrase III (CA3), malate dehydrogenase (MDH1), lactate dehydrogenase B (LDHB), glucose phosphate isomerase (GPI), Hsp70 (HSPA1A), troponin I, fast skeletal muscle (TNNI2), troponin I, cardiac muscle (TNNI3), mitogen-activated protein kinase 12 (MAPK12) and calcium-calmodulin-dependent protein kinase II alpha (CAMK2A). Hsp70, MAPK12 and CAMK2A have not previously been reported to be altered in DMD.

Several proteins that are associated with connective tissue remodeling were also identified, including prolyl endopeptidase FAP (FAP), protein jagged-1 (JAG1), bone sialoprotein 2 (IBSP), ADAM metallopeptidase domain 9 (ADAM9), cadherin-5 (CDH5), neural cell adhesion molecule L1-like protein (CHL1), osteomodulin (OMD) and contactin-5 (CNTN5). These are normally extracellular proteins, and each was found to be significantly lower in DMD group relative to control group at all ages. These proteins may regulate connective remodeling in skeletal muscle.

Certain proteins identified are functionally associated with inflammation and innate immune pathways, including IL34, C-X-C motif chemokine 10 (CXCL10), phospholipase A2, Group IIA (PLA2G2A), hepatoma-derived growth factor-related protein 2 (HDGFRP2), Interleukin-34 (IL34), CD55/Complement decay-accelerating factor/DAF (CD55 or DAF) and RELT tumor necrosis factor receptor (RELT). These members of the inflammatory/innate immune group alternatively demonstrate increased or decreased levels in DMD sera, and do not show significant change as a function of age, with the exceptions of CD55, which decreased as a function of age in DMD and increased in function of age in control group, and fibrinogen, which decreased as a function of age in both DMD and controls.

Persephin, a member of the GDNF family of neurotrophic factors, was also identified in the study. Persephin signals through the RET receptor tyrosine kinase-mitogen-activated protein kinase pathway, and is known to be expressed in skeletal muscle, motor neurons and possibly Schwann cells. Although its role in motor neurons is uncertain, it may be involved in the reinnervation process. Thus the increased levels of persephin and decreased levels of RET that were observed in DMD patients vs. controls (Table 2) may be markers of the ongoing denervation that is occurring. A therapeutic approach that stabilizes the muscle fibers and stabilizes innervation may result in a lowering of persephin levels.

Two other proteins that emerge from this study are of special interest because they are candidate biomarkers to monitor efficacy of anti-inflammatory agents in DMD patients. Phospholipase A2 (PLA2G2A) activity has been reported to be dramatically increased (10-fold) in the skeletal muscle of DMD patients relative to controls and is associated with muscle inflammation, consistent with the high serum levels reported here. CXCL10 is an extracellular chemokine and its elevation in serum could be associated with increased T-cell infiltration in inflamed skeletal muscle.

Five other proteins thought to be involved in muscle regeneration emerged in this study that are initially at similar levels at a young age between DMD and controls, but then decrease as a function of age in DMD while increasing with age in controls: CD55, RELT, GSN, WFIKKN11, and growth differentiation factor-11 (GDF-11).

Recent studies have suggested that exogenous GDF-11 can reverse age-related cardiomyopathy and skeletal muscle deterioration in mice. The findings presented here suggest that GDF-11 is a candidate for ameliorating the cardiomyopathy as well as skeletal muscle deterioration seen in patients with DMD. There are several preclinical and clinical studies aimed at inhibiting GDF-8, a close homolog of GDF-11. It is likely that these GDF-8 inhibitors also inhibit GDF-11. Thus, methods in which GDF-11 is increased in combination with specific inhibition of GDF-8 are specifically contemplated.

The data for the proteins that are high early in life for DMD patients and which diminish in blood as muscle mass decreases (Group 1; FIG. 2A) suggest that significant muscle cell death is occurring very early in life, perhaps even during embryonic development (a time for which data are not available). Surprisingly, the absence of dystrophin does not cause abrupt muscle cell death: the decrease observed suggests that the number of muscle cells in DMD patients decreases by a median half-life of ˜7.2 years. This observation suggests that there is a balance between muscle stem cell-derived muscle mass preservation and dystrophin-less-derived muscle loss, and the relative “slowness” of muscle cell death might provide an opportunity for a novel non-dystrophin-centric treatment option for DMD patients.

### Example 4

### Treatment of DMD

Several animal models are useful with the methods and compositions of the invention for identifying, modulating and monitoring drug targets in muscular disease. Male mice (e.g., MDx strains) have been maintained without a functional dystrophin. While these mice are not normal, the phenotype is not as severe as the phenotypes of DMD patients. The MDx mouse model becomes more severe and more like the human disease when a second knock-out is added to the dystrophin mutation (a common second mutation is in the utrophin gene). Thus, in one embodiment, GDF-11 can be administered to subject (e.g. mouse model of DMD) in order to ameliorate the symptoms of the subject (e.g., DMD symptoms of the MDx mouse and MDx-utrophin-less mouse. One of ordinary skill in the art knows well method for identifying a therapeutically effective dose. For example, it is possible to first analyze the required GDF-11 injection doses and injection schedule to maintain the circulating GDF-11 concentration at or near a wild-type level, and the determined dose could be used in the dystrophin and dystrophin-utrophin models. In addition, dog and pig dystrophin knock-outs can also be treated with injected GDF-11.

For humans, dosing pharmacokinetics and safety can be established. After preclinical safety/toxicity experiments have been completed to regulatory standards, a drug concentration is identified at which toxicity starts, and the target organs for toxicity identified. In one non-limiting example, human experiments are performed in single escalating dose experiments followed by multiple dose escalation experiments, usually in healthy volunteers although in this case it might be better done in DMD subjects depending on discussion with an IRB and with parent organizations because the pharmacokinetics (PK) in 18-45 year old healthy volunteers might be different. If required by such discussions, the PK experiments might need to be performed in healthy adults first and then confirmed in smaller groups of DMD children. For single dose, groups of 8 subjects (randomized to 8 active and 2 placebo per group) receive a subcutaneous and/or intramuscular injection. Blood samples are taken in a time series, for example, at 0, 0.5, 1, 2, 4, 8, 24, 48 hours after the injection. Doses would be calculated using the mouse pharmacology and toxicity data to start at a level below any active level, and the PK and safety checked in each group before the next escalation. Subsequent groups often go up in half log dose steps until adverse effects are experienced or until a predefined stopping rule for a concentration. Typically 6 or more dose escalations are performed before a limiting adverse effect but this can be dependent upon the pharmacology.

Multiple dose studies are similar in group size and usually last 2 weeks to establish safety and steady-stake PK. These studies may use the single dose experiments' information as a starting point so the initial dose is likely to be higher. Using the PK results from single dose, a dosing regimen can be defined which is likely to achieve a target concentration or which ensures that it does not fall below a defined trough. This may be once, twice or three times a day. If there is uncertainty, the multiple dose experiment might use more than one dosing regimen. Initially if the PK is short, dosing regimens can be used which would not be practical on a large scale but which will test the hypothesis; if efficacy is achieved PK can be improved and regimens made more practical through slow release formulations.

Efficacy experiments can be performed in subjects with DMD using the regimens identified in the multiple dose PK study which achieved the target concentration (e.g. matching the normal concentration or higher). Typically a phase IIa efficacy experiment would test placebo plus 2-3 doses and dosing regimens. Groups may be of the order of 20 subjects each, selected to be early enough in the disease such that improvement is possible, and the study duration would be estimated to be long enough to see trends efficacy differences, not necessarily with each group reaching statistically significant—this may be 3-6 months or an adaptive design could be used where a data safety monitoring board lets the study continue until either futility or a difference is apparent. Metrics for efficacy may include 6 minute walk, muscle MRI, muscle biopsy and blood based biomarkers using SOMAscan and/or immunoassays. Trends in the right direction would lead to a phase IIb program which would use the phase IIa metrics to define a statistically powered size and duration. If the dosing regimen required is impractical, slow release formulations would be developed, go through the single and multiple dose PK and then into phase IIb.

Various modification, recombination, and variation of the described features and embodiments will be apparent to those skilled in the art without departing from the scope and spirit of the invention. Although specific embodiments have been described, it should be understood that the invention as claimed should not be unduly limited to such specific embodiments. Indeed, various modifications of the described modes and embodiments that are obvious to those skilled in the relevant fields are intended to be within the scope of the following claims. All publications and patents mentioned in the present application and/or listed below are herein incorporated by reference in their entireties.

### REFERENCES

1. Bushby K, et al. (2010) Diagnosis and management of Duchenne muscular dystrophy, part 1: diagnosis, and pharmacological and psychosocial management. The Lancet. Neurology 9(1):77-93.
2. Mah J K, et al. (2014) A systematic review and meta-analysis on the epidemiology of Duchenne and Becker muscular dystrophy. Neuromuscular disorders: NMD 24(6):482-491.
3. Ervasti J M & Campbell K P (1993) A role for the dystrophin-glycoprotein complex as a transmembrane linker between laminin and actin. The Journal of cell biology 122(4):809-823.
4. Hoffman E P, Brown R H, Jr., & Kunkel L M (1987) Dystrophin: the protein product of the Duchenne muscular dystrophy locus. Cell 51(6):919-928.
5. Wong B L & Christopher C (2002) Corticosteroids in Duchenne muscular dystrophy: a reappraisal. Journal of child neurology 17(3):183-190.
6. Hoffman E P, et al. (2012) Novel approaches to corticosteroid treatment in Duchenne muscular dystrophy. Physical medicine and rehabilitation clinics of North America 23(4):821-828.
7. Manzur A Y, Kuntzer T, Pike M, & Swan A (2008) Glucocorticoid corticosteroids for Duchenne muscular dystrophy. The Cochrane database of systematic reviews (1):CD003725.
8. Cirak S, et al. (2011) Exon skipping and dystrophin restoration in patients with
Duchenne muscular dystrophy after systemic phosphorodiamidate morpholino oligomer treatment: an open-label, phase 2, dose-escalation study. Lancet 378(9791):595-605.
9. Goemans N M, et al. (2011) Systemic administration of PRO051 in Duchenne's muscular dystrophy. The New England journal of medicine 364(16):1513-1522.
10. Mendell J R, et al. (2013) Eteplirsen for the treatment of Duchenne muscular dystrophy. Annals of neurology 74(5):637-647.
11. Mendell J R, et al. (2010) Dystrophin immunity in Duchenne's muscular dystrophy. The New England journal of medicine 363(15):1429-1437.
12. Jarmin S, Kymalainen H, Popplewell L, & Dickson G (2014) New developments in the use of gene therapy to treat Duchenne muscular dystrophy. Expert opinion on biological therapy 14(2):209-230.
13. Peltz S W, Morsy M, Welch E M, & Jacobson A (2013) Ataluren as an agent for therapeutic nonsense suppression. Annual review of medicine 64:407-425.
14. Fairclough R J, Wood M J, & Davies K E (2013) Therapy for Duchenne muscular dystrophy: renewed optimism from genetic approaches. Nature reviews. Genetics 14(6):373-378.
15. Heier C R, et al. (2013) VBP15, a novel anti-inflammatory and membrane-stabilizer, improves muscular dystrophy without side effects. EMBO molecular medicine 5(10):1569-1585.
16. Leung D G & Wagner K R (2013) Therapeutic advances in muscular dystrophy. Annals of neurology 74(3):404-411.
17. McDonald C M, et al. (2013) The 6-minute walk test and other endpoints in Duchenne muscular dystrophy: longitudinal natural history observations over 48 weeks from a multicenter study. Muscle & nerve 48 (3):343-356.
18. McDonald C M, et al. (2010) The 6-minute walk test as a new outcome measure in Duchenne muscular dystrophy. Muscle & nerve 41 (4):500-510.
19. Anderson N L (2010) The clinical plasma proteome: a survey of clinical assays for proteins in plasma and serum. Clinical chemistry 56(2):177-185.
20. Rifai N, Gillette M A, & Carr S A (2006) Protein biomarker discovery and validation: the long and uncertain path to clinical utility. Nature biotechnology 24(8):971-983.
21. Hathout Y, et al. (2014) Discovery of serum protein biomarkers in the mdx mouse model and cross-species comparison to Duchenne muscular dystrophy patients. Human molecular genetics 23(24):6458-6469.
22. Ayoglu B, et al. (2014) Affinity proteomics within rare diseases: a BIO-NMD study for blood biomarkers of muscular dystrophies. EMBO molecular medicine 6(7):918-936.
23. Gold L, et al. (2010) Aptamer-based multiplexed proteomic technology for biomarker discovery. PloS one 5(12):e15004.
24. Gold L, Walker J J, Wilcox S K, & Williams S (2012) Advances in human proteomics at high scale with the SOMAscan proteomics platform. New biotechnology 29(5):543-549.
25. Rohloff J C, et al. (2014) Nucleic Acid Ligands With Protein-like Side Chains: Modified Aptamers and Their Use as Diagnostic and Therapeutic Agents. Molecular therapy. Nucleic acids 3:e201.
26. Jaszai J, et al. (1998) GDNF-related factor persephin is widely distributed throughout the nervous system. Journal of neuroscience research 53(4):494-501.
27. Lindahl M, Backman E, Henriksson K G, Gorospe J R, & Hoffman E P (1995) Phospholipase A2 activity in dystrophinopathies. Neuromuscular disorders: NMD 5(3):193-199.
28. Kim J, et al. (2014) Therapeutic effect of anti-C-X-C motif chemokine 10 (CXCL10) antibody on C protein-induced myositis mouse. Arthritis research & therapy 16 (3):R126.
29. Loffredo F S, et al. (2013) Growth differentiation factor 11 is a circulating factor that reverses age-related cardiac hypertrophy. Cell 153(4):828-839.
30. Lee Y S & Lee S J (2013) Regulation of GDF-11 and myostatin activity by GASP-1 and GASP-2. Proceedings of the National Academy of Sciences of the United States of America 110(39):E3713-3722.
31. Smith R C & Lin B K (2013) Myostatin inhibitors as therapies for muscle wasting associated with cancer and other disorders. Current opinion in supportive and palliative care 7(4):352-360.
32. Benjamini Y & Hochberg Y (1997) Controlling the false discovery rate: a practical and powerful approach to multiple testing. J. Roy. Statist. Soc. Ser. B 57:289-300.

1. A method for treating a subject for muscular dystrophy comprising administering, to a subject in need, a therapeutic effective amount of a therapeutic agent selected from GDF-11, RELT, CD55, WFIKKN1, gelsolin, fibroblast activation protein alpha (FAP), protein jagged-1 (JAG1), bone sialoprotein 2 (IBSP), ADAM metallopeptidase domain 9 (ADAM9), cadherin-5 (CDH5), neural cell adhesion molecule L1-like protein (CHL1), osteomodulin (OMD), contactin-5 (CNTN5), and combinations thereof.

2. A method for treating a subject for muscular dystrophy comprising administering, to a subject in need, a therapeutic effective amount of a protein analog or agonist of GDF-11, RELT, CD55, WFIKKN1, gelsolin, fibroblast activation protein alpha (FAP), protein jagged-1 (JAG1), bone sialoprotein 2 (IBSP), ADAM metallopeptidase domain 9 (ADAM9), cadherin-5 (CDH5), neural cell adhesion molecule L1-like protein (CHL1), osteomodulin (OMD), and/or contactin-5 (CNTN5).

3. The method of claim 1 or claim 2, wherein the muscular dystrophy is Duchenne Muscular Dystrophy.

4. The method of any one of claims 1 to 3, wherein the administration of the therapeutic agent to the subject thereby relieves, improves and/or reduces the symptoms of muscular dystrophy in the subject.

5. The method of claim 4, wherein the administration improves muscle strength and/or increases muscle mass in the subject.

6. The method of any one of claims 1 to 5, wherein the method comprises administering GDF-11.

7. The method of any one of claims 1 to 6, wherein the method further comprises administering an antagonist of GDF-8.

8. A method for determining a treatment course of action, comprising:
a) assaying a tissue sample from a subject diagnosed with muscular disease to identify altered levels of one or more proteins relative to the level of said proteins in normal tissue; and
b) administering one or more treatments that targets one or more of said proteins with altered expression.

9. The method of claim 8, wherein said proteins are selected from GDF-11, RELT, CD55, WFIKKN1, gelsolin, fibroblast activation protein alpha (FAP), protein jagged-1 (JAG1), bone sialoprotein 2 (IBSP), ADAM metallopeptidase domain 9 (ADAM9), cadherin-5 (CDH5), neural cell adhesion molecule L1-like protein (CHL1), osteomodulin (OMD), and contactin-5 (CNTN5), and combinations thereof.

10. The method of claim 8 or claim 9, wherein said reference sample is sample of normal tissue from said subject, or a population average of normal tissue.

11. The method of any one of claims 8 to 10, wherein the level of said protein is altered at least 4-fold relative to the level in said reference sample.

12. The method of any one of claims 8 to 10, wherein the level of said protein is altered at least 50-fold relative to the level in said reference sample.

13. The method of any one of claims 8 to 12 further comprising administering said one or more treatments to said subject.

14. The method of any one of claims 8 to 13, further comprising the step of determining the presence of mutations in said proteins.

15. The method of any one of claims 8 to 14, wherein said disease is a muscular disease.

16. The method of any one of claims 8 to 15, wherein the biological sample is selected from the group consisting of tissue, whole blood, leukocytes, peripheral blood mononuclear cells, buffy coat, plasma, serum, sputum, tears, mucus, nasal washes, nasal aspirate, breath, urine, semen, saliva, peritoneal washings, ascites, cystic fluid, meningeal fluid, amniotic fluid, glandular fluid, pancreatic fluid, lymph fluid, pleural fluid, cytologic fluid, nipple aspirate, bronchial aspirate, bronchial brushing, synovial fluid, joint aspirate, organ secretions, cells, a cellular extract and cerebrospinal fluid.

17. The method of any one of claims 8 to 16, wherein said assaying comprises contacting said sample with a plurality of aptamers specific for said proteins.

18. A method for determining a treatment course of action, comprising:
a) assaying a tissue sample from a subject diagnosed with muscular disease to identify altered levels of one or more proteins selected from GDF-11, RELT, CD55, WFIKKN1, gelsolin, fibroblast activation protein alpha (FAP), protein jagged-1 (JAG1), bone sialoprotein 2 (IBSP), ADAM metallopeptidase domain 9 (ADAM9), cadherin-5 (CDH5), neural cell adhesion molecule L1-like protein (CHL1), osteomodulin (OMD), and contactin-5 (CNTN5) relative to the level of said proteins in normal tissue; and
b) administering one or more treatments that targets one or more of said proteins with altered expression.

19. The method of claim 18, wherein the level of said proteins are altered at least 4-fold relative to the level in normal tissue.

20. The method of claim 18 or claim 19, wherein the level of said proteins are altered at least 50-fold relative to the level in normal tissue.

21. The method of any one of claims 18 to 20, further comprising the step of determining the presence of mutations in said proteins.

22. The method of any one of claims 18 to 21, wherein said assaying comprises contacting said sample with a plurality of aptamers specific for said proteins.

23. A method for treating a disease, comprising:
a) assaying a biological sample from a subject diagnosed with a disease to identify altered levels of one or more proteins relative to the level of said protein in a reference sample; and
b) administering one or more treatments that target one or more of said proteins with altered expression to said subject.

24. The method of claim 23, wherein said proteins are selected from GDF-11, RELT, CD55, WFIKKN1, gelsolin, fibroblast activation protein alpha (FAP), protein jagged-1 (JAG1), bone sialoprotein 2 (IBSP), ADAM metallopeptidase domain 9 (ADAM9), cadherin-5 (CDH5), neural cell adhesion molecule L1-like protein (CHL1), osteomodulin (OMD), and contactin-5 (CNTN5).

25. The method of claim 23 or claim 24, wherein said reference sample is sample of normal tissue from said subject, or a population average of normal tissue.

26. The method of any one of claims 23 to 25, wherein the level of said protein is altered at least 2-fold relative to the level in said reference sample.

27. The method of any one of claims 23 to 26, wherein the level of said protein is altered at least 50-fold relative to the level in said reference sample.

28. The method of any one of claims 23 to 27, further comprising the step of determining the presence of mutations in said proteins.

29. The method of any one of claims 23 to 28, wherein said disease is muscular disease.

30. The method of claim 29, wherein the muscular disease it Duchene muscular dystrophy.

31. The method of any one of claims 23 to 30, wherein the biological sample is selected from the group consisting of tissue, whole blood, leukocytes, peripheral blood mononuclear cells, buffy coat, plasma, serum, sputum, tears, mucus, nasal washes, nasal aspirate, breath, urine, semen, saliva, peritoneal washings, ascites, cystic fluid, meningeal fluid, amniotic fluid, glandular fluid, pancreatic fluid, lymph fluid, pleural fluid, cytologic fluid, nipple aspirate, bronchial aspirate, bronchial brushing, synovial fluid, joint aspirate, organ secretions, cells, a cellular extract and cerebrospinal fluid.

32. A method for monitoring treatment of a disease, comprising:
a) assaying a biological sample from a subject diagnosed with a disease to identify altered levels of one or more proteins relative to the level of said protein in a reference sample;
b) administering one or more treatments that target one or more of said proteins with altered expression to said subject; and
c) repeating step a) one or more times.

33. The method of claim 32, wherein the one or more proteins are each independently selected from the proteins in Table 2.

34. The method of claim 32 or claim 33, wherein the one or more treatments are selected from GDF-11, RELT, CD55, WFIKKN1, gelsolin, fibroblast activation protein alpha (FAP), protein jagged-1 (JAG1), bone sialoprotein 2 (IBSP), ADAM metallopeptidase domain 9 (ADAM9), cadherin-5 (CDH5), neural cell adhesion molecule L1-like protein (CHL1), osteomodulin (OMD), and contactin-5 (CNTN5).

35. The method of any one of claims 32 to 34, wherein the disease is muscular disease.

36. The method of claim 35, wherein the disease is Duchene muscular dystrophy.

37. A method for monitoring progression of a muscular disease, comprising assaying a biological sample from a subject diagnosed with a disease to identify altered levels of one or more proteins listed in Table 2 relative to the level of said protein in a reference sample.

38. A method for monitoring progression of a muscular disease, comprising assaying a first biological sample from a subject with a muscular disease and assaying a second biological sample from the subject, wherein the first and second biological samples were taken at a first time point and a second time point, to identify altered levels of one or more proteins listed in Table 2 at the second time point relative to the first time point.

39. The method of claim 37 or claim 38, wherein at least one protein is selected from GDF-11, RELT, CD55, WFIKKN1, gelsolin, fibroblast activation protein alpha (FAP), protein jagged-1 (JAG1), bone sialoprotein 2 (IBSP), ADAM metallopeptidase domain 9 (ADAM9), cadherin-5 (CDH5), neural cell adhesion molecule L1-like protein (CHL1), osteomodulin (OMD), contactin-5 (CNTN5), HSPA1A, MAPK12, CAMK2A, CXCL10, RET, and persephin.

40. The method of claim 39, wherein a decrease in the level of at least one protein selected from GDF-11, RELT, CD55, WFIKKN1, gelsolin, fibroblast activation protein alpha (FAP), protein jagged-1 (JAG1), bone sialoprotein 2 (IBSP), ADAM metallopeptidase domain 9 (ADAM9), and cadherin-5 (CDH5), neural cell adhesion molecule L1-like protein (CHL1), osteomodulin (OMD), and contactin-5 (CNTN5) at the second time point relative to the first time point, or an increase in the levels of at least one protein selected from HSPA1A, MAPK12, CAMK2A, CXCL10, RET, and persephin at the second time point relative to the first time point, indicates that the muscular disease has progressed between the first time point and the second time point.

41. The method of claim 39, wherein an increase in the level of at least one protein selected from GDF-11, RELT, CD55, WFIKKN1, gelsolin, fibroblast activation protein alpha (FAP), protein jagged-1 (JAG1), bone sialoprotein 2 (IBSP), ADAM metallopeptidase domain 9 (ADAM9), and cadherin-5 (CDH5) at the second time point relative to the first time point, or a decrease in the levels of at least one protein selected from contactin-5 (CNTN5), CXCL10, RET, and persephin at the second time point relative to the first time point, indicates that the muscular disease has improved between the first time point and the second time point.

42. The method of any one of claims 37 to 41, wherein the muscular disease is Duchene muscular dystrophy.

43. The method of any one of claims 38 to 42, wherein the first and second biological samples were taken 1 week, 2 weeks, 1 month, 2 months, 3 months, 4 month, 5 months, 6 months, 1 year, or more than 1 year apart.

44. The method of any one of claims 37 to 43, wherein the biological sample is selected from the group consisting of tissue, whole blood, leukocytes, peripheral blood mononuclear cells, buffy coat, plasma, serum, sputum, tears, mucus, nasal washes, nasal aspirate, breath, urine, semen, saliva, peritoneal washings, ascites, cystic fluid, meningeal fluid, amniotic fluid, glandular fluid, pancreatic fluid, lymph fluid, pleural fluid, cytologic fluid, nipple aspirate, bronchial aspirate, bronchial brushing, synovial fluid, joint aspirate, organ secretions, cells, a cellular extract and cerebrospinal fluid.

45. The method of any one of claims 37 to 44, wherein said assaying comprises contacting said sample with a plurality of aptamers specific for said proteins.
